(19) United States
(12) Patent Application Publication
(10) Pub. No.: US 2019/0308035 A1
(43) Pub. Date: Oct. 10, 2019

(54) A NON-INVASIVE METHOD FOR SELECTIVE NEURAL STIMULATION BY ULTRASOUND

(71) Applicant: The Hong Kong Polytechnic University, Hong Kong (HK)

(72) Inventors: Lei Sun, Shatin (HK); Zhihai Qiu, Longyan, Shanghang City (CN); Yaoheng Yang, Shanghai (CN); Jinghui Guo, Shatin (HK); Shashwati Kala, Peng Chau (HK); Jiejun Zhu, Zhangzhou City (CN)

(21) Appl. No.: 15/949,991

(22) Filed: Apr. 10, 2018

(51) Int. Cl. A61N 7/00; A61K 49/00; A61K 41/00; C12N 13/00; C12N 5/0793

(52) U.S. Cl. CPC A61N 7/00; A61K 49/0017; A61N 2007/0026; C12N 13/00; C12N 5/0619; A61K 41/0047

(57) ABSTRACT

In an aspect of the present disclosure there is provided a method for reversibly stimulating neuronal cells, said method comprising activating mechanosensitive ion channels of neuronal cells expressing said channels by exposing said cells to an ultrasound stimulus.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 900220_408_SEQUENCE_LISTINGtxt. The text file is 12.1 KB, was created on Jul. 7, 2018, and is being submitted electronically via EFS-Web.

The present disclosure relates to non-invasive methods and mechanisms of selectively manipulation of neuronal activity in specific spatio-temporal regions using ultrasound.

### BACKGROUND

In the brain, billions of neurons work together as an intricately organized, interconnected circuits, supporting the vast diversity of animal behaviour, up to advanced functions like consciousness, cognition, and emotion. These neural circuits are both extremely complex and exquisitely specific. It would be appreciated that controlling local or global neural activity and signalling by physical intervention is a powerful way to gain causal insight into brain functions and treat brain disorders.

Diverse modalities have been developed in the past few decades in an attempt to control such neural circuits, ranging from highly invasive deep brain stimulation (DBS), to less invasive transcranial direct current stimulation (tDCS), transcranial magnetic stimulation (TMS), chemogenetics and optogenetics.

Stimulation modalities with less invasiveness or non-invasiveness, such as transcranial magnetic stimulation (TMS), transcranial direct current stimulation (tDCS) etc., have been utilized to treat brain disease and dysfunction. These have demonstrated various advantages and have great value in clinical practice, but lack spatiotemporal resolution. Furthermore, these modalities requires precise identification and pre-tagging of neural circuits for subsequent selective stimulation, and it is difficult to assess the basal conditions of these circuits before activation.

Another potentially promising area is optogenetics. Optogenetics is based on artificial inserted well-characterized light sensitive proteins (e.g. opsins) to chosen neurons, which are enabled to respond to light stimulation, while leaving other cells non responsive even when receiving the same dose of light stimulation. However, the limited penetration depth of light in brain tissue means the stimulation is constrained to a small region. Also the light based stimulation can have some undesired side-effects due to its invasiveness or miss circuits in the deep brain. Optogenetics is hard to translate to clinical use.

Ultrasound-based brain stimulation is a promising candidate because it can potentially access deep brain structures non-invasively through the intact skull and be steered to millimetre-sized focal spots with brain-wide scalability [1], making it potentially translatable to clinical routines.

However, there are technical and practical challenges in the following aspects:

First, governed by diffraction limit of acoustic wave, ultrasound at current applied frequencies can only be focused into a millimetre size spot. By using novel beam focusing and image-guided technology, compensation for the heterogeneity of brain tissue to form sub-diffraction pattern is possible, however, the focal spot is still too large for single neuron or neuron type stimulation.

Second, the underlying mechanism of ultrasound neuro-stimulation is still unclear, thus it is hard to make this stimulation controllable and optimize the stimulation outcome. In particular, there is not enough selectivity to pinpoint specific targeted neurons to stimulate the chosen neurons involved in specific circuits of the brain.

Thus, the lack of cell-type or circuit-element selectivity is a barrier to successful application of ultrasound based brain stimulation.

There is a need therefore to provide a non-invasive method for stimulation of selected neural circuits for understanding brain function and treating brain disorders with high spatial and temporal precision.

### SUMMARY

Features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims.

The present disclosure provides a method for ultrasound stimulation of mechanosensitive ion channels in selected neural circuits for inducing neural activity with high spatial and temporal resolution.

Advantageously, in one aspect, there is provided a method for reversibly stimulating at least one or more neuronal cells, said method comprising activating mechanosensitive ion channels of at least one or more neuronal cells expressing said channels by exposing said cells to an ultrasound stimulus.

Preferably, the mechanosensitive ion channels are selected from the group comprising Piezo 1, MscL-G22s, CFTR

Advantageously, mechanosensitive ion channels expressed in the neuronal cells may be introduced to or increased in said cells by introducing a recombinant nucleic acid encoding the mechanosensitive ion channels into said cells or precursors thereof.

The mechanosensitive ion channels may be introduced to or increased in the neuronal cells by transfection with a plasmid or infection with a virus containing a promoter sequence coupled with a mechanosensitive ion channel gene.

The ultrasound stimulus may be in the range of 0.1 MPa to 0.6 MPa, 0.2 MPa to 0.5 MPa, or optionally 0.5 MPa.

The ultrasound stimulus applied may be at about 500 kHz ultrasound of at least 200 cycles, at about 1 kHz pulse repetition frequency (PRF) with 200 tone bursts at about 0.1, 0.2, and 0.3 MPa.

The method may further comprise monitoring the intracellular cation activity, which may be intracellular Ca2+ activity.

The step of determining intracellular Ca2+ activity may be performed by any one or more of fluorescent staining, levels of Calcium/calmodulin-dependent protein kinase type II (p-CaMKII) and the transcription factor CREB (p-CREB).

Optical monitoring of intracellular Ca2+ activity in vivo in an animal may be conducted at least during the application of the ultrasound stimulus to the animal.

The optical monitoring may be performed by an image capturing device engageable with the animal.

According to a second aspect of the disclosure there is provided a method for studying specific spatial and temporal activity of neuron cells in vivo in a mammal comprising:

(a) transfecting neuronal cells with plasmids having one or more sequences encoding a mechanosensitive ion channel into neuronal cells to produce infected neuronal cells expressing increased numbers of said channel; and

(b) exposing said cells to an ultrasound stimulus whilst monitoring the intracellular Ca2+ activity therein.

In a further aspect of the disclosure there is provided a method of increasing neuron sensitivity to ultrasound by overexpression therein of at least one or more proteins selected from the group comprising piezol, MscL-G22s, CFTR, wherein the overexpression is by introducing genetic material encoding the at least one or more proteins in the neuronal cells by transfection with a plasmid or a virus wherein said plasmid or virus has a sequence encoding the at least one or more proteins.

In yet another aspect of the disclosure there is provided a system for studying specific spatial and temporal activity of neuron cells in vivo in a mammal, the system comprising:

a population comprising a plurality of neuronal cells genetically modified so as to express therein an increased number of mechanosensitive ion channels;

an ultrasound source for activating mechanosensitive ion channels engageable with the mammal and proximal to said cells;

an optical imaging source arranged adjacent the transfected neuronal cells for providing images of the neuronal cells at least during activation of the mechanosensitive ion channels by the ultrasound source.

Preferably, the ultrasound source activates the mechanosensitive ion channels in the population.

### BRIEF DESCRIPTION OF THE DRAWINGS

In order to describe the manner in which the above-recited and other advantages and features of the disclosure can be obtained, a more particular description of the principles briefly described above will be rendered by reference to specific embodiments thereof which are illustrated in the appended Figures. Understanding that these Figures depict only exemplary embodiments and are not therefore to be considered to be limiting of its scope, the principles herein are described and explained with additional specificity and detail.

Preferred embodiments of the present disclosure will be explained in further detail below by way of examples and with reference to the accompanying Figures, in which:—

FIG. 1A depicts a schematic representation of the experimental schema of an aspect of the present disclosure in which the cells are sensitised to ultrasound stimulation by inducing the expression of mechanosensitive ion channels such as Piezo1.

FIG. 1B illustrates Piezo1 overexpression in 293T cells transfected with the Piezo1-containing plasmid, as evaluated by Western blot and RT-qPCR.

FIG. 1C demonstrates ultrasound induced dose-dependent calcium influx in 293T cells overexpressing Piezo1. Ultrasound stimulating mode: each stimulus contained 200 tone bursts of 500 kHz ultrasound at 1 kHz RPF, 200 cycles, and 0.1 MPa, 0.2 MPa, and 0.3 MPa.

FIG. 1D depicts immuncytochemical staining ultrasound stimulation induced significant increase of intracellular calcium of the neuron, comparable with the effect of Yodal, a chemical agonist of Piezo1.

FIG. 1E depicts immunocytochemical staining showing primary neurons made to overexpress Piezo1 by plasmid transfection accumulate more intracellular calcium when treated with ultrasound than do neurons treated with a control plasmid.

FIG. 1F demonstrates functional validation of Piezo1 expression in 293T cells wherein Yodal, a Piezo1 agonist, induces significantly higher calcium influx in 293T cells overexpressing Piezo1 than in control cells.

FIG. 2A depicts the expression of Piezo1 in CLU199 cells, evaluated by RT-PCR, with HeLa cells shown as a positive control.

FIG. 2B depicts a Western Blot of in CLU199 cells showing that ultrasound significantly upregulates expression of key CA2+ signalling regulators p-CamKII and p-CREB in a dose-dependent manner.

FIG. 2C shows ultrasound and Piezo1 agonist Yodal (1 μM) both upregulate p-CamKII and p-CREB to a similar degree. Using the Piezo1-specific blocker GsMTx-4 (10 μM) inhibits this effect, shown by a Western blot.

FIG. 2D depicts immunocytochemical staining for an siRNA knockdown of Piezo1 in CLU199 shown by RT-qPCR.

FIG. 2E shows how the Piezo1 knockdown of FIG. 2D decreases the effect of ultrasound on the expression of p-CamKII and p-CREB.

FIG. 2F is a graph showing the number of cell nuclei showing c-Fos staining and how ultrasound treatment significantly activates c-Fos expression in primary cortical neurons.

FIG. 2G Results of a RT-qPCR array probing genes regulated by calcium signalling. The pie chart shows the total number of genes upregulated or downregulated by ultrasound. The Venn diagrams show the number of genes upregulated and downregulated by ultrasound compared to Yodal.

FIG. 2H is a is a graph showing genes from the array which have an expression level which was altered by two times or more by ultrasound (US) shown along with the levels of the same genes in the other treatment conditions. Results are expressed as log 10 fold change compared to the untreated control.

FIG. 2I is immunocytochemical staining in primary cortical neurons untreated, treated by ultrasound or treated with GsMTx-4 (10 μM) followed by ultrasound showing how ultrasound regulates the activity of calcium-related signalling in primary cortical neurons.

FIG. 2J shows the effects of ultrasound, blocking Piezo1 before ultrasound and a Piezo1 agonist on gene expression.

FIG. 3A depicts an experimental schema for the in vivo study in which mouse brains were made to overexpress Piezo1 by injecting plasmid locally into the M1 (motor cortex) region.

FIG. 3B is a graph showing a higher rate of tail flicks is induced by ultrasound stimulation (0.1-0.6 MPa) in Piezo1-transfected mice than that of control mice under anaesthesia.

FIG. 3C depicts a graph of results showing that ultrasound (0.3 MPa) induced a higher rate of head movement in Piezo1-transfected mice than in control mice when moving freely.

FIG. 3D is a graph and an immunocytochemical staining showing c-Fos expression after ultrasound stimulation is significantly higher in Piezo1-transfected mice than in control mice.

FIG. 4A shows expression of MscL-G22s EYFP in neurons.

FIG. 4B depicts patch clamping results of control neurons relative to MscL expressing neurons using 0.05 MPA ultrasound stimulation (shaded region).

FIG. 4C is a graph showing the response rate of MscL transfected mice brain when stimulated with ultrasound relative to a control under the same stimulation conditions.

FIG. 5A is a schematic representation of an embodiment of a calcium imaging system and an ultrasound stimulation system.

FIG. 5B is an exemplary schematic representation of how calcium imaging can be conducted in vivo simultaneously with the application of ultrasound which assists in understanding outcomes of opening particular mechanosensitive ion channels in specific portions of the mouse brain.

FIG. 5C depicts an exemplary image captured from the arrangement depicted in FIG. 5B.

FIG. 6A illustrates an exemplary genemap of Piezo1-EGFP.

FIG. 6B illustrates an exemplary genemap of Piezo1::GCaMP6s.

FIG. 6C illustrates an exemplary genemap of MscL-EYFP.

FIG. 6D illustrates an exemplary genemap of MscL:: GCaMP6s.

### DETAILED DESCRIPTION OF PREFERRED EMBODIMENTS

### Definitions

“Mechanosensitive” ion channels, are a component of the cellular force-sensation machinery whose opening is controlled by diverse mechanical stimuli such as touch, hearing, crowding, stretch and cell volume, which effectively “translates” physical force into chemical messages [2].

“Expressing mechanosensitive ion channels” means the production of one or more mechanically activatable molecular channel proteins into a cell in which a recombinant nucleic acid molecule encoding the mechanically activated molecular channel protein has been introduced by genetic means.

“Transgenic methods” is meant a method of introduction of recombinant nucleic acid molecules into the genome of the organism.

“Defined spatial region” is meant a predetermined specific and confined area in the brain.

A preferred embodiment for genetically targeted expression of the mechanosensitive proteins of the present disclosure comprises a vector which contains the gene for such protein.

As used herein, the term “vector” refers to a nucleic acid molecule capable of transporting another nucleic acid to which it has been operatively linked between different genetic environments.

The term “vector” also refers to a virus or organism capable of transporting nucleic acid molecules. Preferred vectors are can replicate/express the nucleic acids to which they are linked. Vectors capable of directing the expression of genes to which they are operatively linked are referred to herein as “expression vectors”. Other preferred vectors include viruses such as recombinant AAV virus. Preferred vectors can genetically insert mechanosensitive proteins into both dividing and non-dividing cells; in-vivo or in-vitro.

Those vectors can include a prokaryotic promoter capable of directing the expression (transcription and translation) of the protein in a bacterial host cell, such as E. coli. A promoter is an expression control element formed by a DNA sequence that permits binding of RNA polymerase and transcription to occur. Promoter sequences compatible with bacterial hosts are typically provided in plasmid vectors containing convenient restriction sites for insertion of a DNA segment according to an aspect of the present disclosure.

Expression vectors compatible with eukaryotic cells, can also be used. Eukaryotic cell expression vectors are well known and available from several commercial sources. Typically, such vectors are provided containing convenient restriction sites for insertion of the desired DNA homologue.

One preferred embodiment of an expression vector of the present disclosure is a rAAV virus comprising the gene for the mechanosensitive ion channel together with a hSyn promoter. This vector is used in one aspect of the present disclosure to deliver genes to specific neurons.

Various embodiments of the disclosure are discussed in detail below. While specific implementations are discussed, it should be understood that this is done for illustration purposes only. A person skilled in the relevant art will recognize that other components and configurations may be used without departing from the scope of the disclosure.

The use of ultrasound triggered mechanosensitive ion channels as described herein addresses the need in the art for non-invasive, precisely controllable, wide field of view stimulation of neurons and neural circuits for understanding advanced brain function and developmental problems.

The present disclosure identified ultrasound-sensitive cellular machinery that can respond to stimulation and consequently initiate neural activity and signalling, granting precise control of mammalian neurons and animal behaviour. “Mechanosensitive” ion channels, are a component of the cellular force-sensation machinery whose opening is controlled by diverse mechanical stimuli such as touch, hearing, crowding, stretch and cell volume, which effectively “translates” physical force into chemical messages [2].

The piezo-type mechanosensitive ion channel component 1 (Piezo1) is a cation channel and is reported to exhibit a preference for calcium ions (Ca2+) [3], whose intracellular levels have been widely used as an indicator for neural activation (reviewed in [4]).

Similarly, MscL is a bacterial originated mechanosensitive ion channel, named: The Large Conductance Mechanosensitive Ion Channel (MscL) Family.

The estimated force required to activate Piezo1 can be as low as 10 pN.

As used herein the terms Piezo 1 and MscL-G22s are taken to mean the full proteins or fragments thereof. Further, the Piezo1 protein of the present disclosure also comprises the protein sequence from DNA sequence of SEQ ID NO:1. The MscL protein of the present disclosure also comprises the protein sequence from DNA Sequence of SEQ ID NO:2.

“Protein” in this sense includes proteins, polypeptides, and peptides. Also included within the protein of the present disclosure are amino acid variants of the naturally occurring sequences, as determined herein. Preferably, the variants are greater than about 75% homologous to the protein sequence of Piezo1, MscL, and more preferably greater than about 80%, even more preferably greater than about 85% and most preferably greater than 90%. In some embodiments the homology will be higher.

Homology means sequence similarity or identity, with identity being preferred. This homology will be determined using standard techniques.

The present disclosure includes nucleic acid sequences provided herein including variants which are more than about 65% homologous to the provided sequence, more than about 70% homologous to the provided sequence, more than about 75% homologous to the provided sequence, more than about 80% homologous to the provided sequence, more than about 85% homologous to the provided sequence, more than about 90% homologous to the provided sequence, or more than about 95% homologous to the provided sequence.

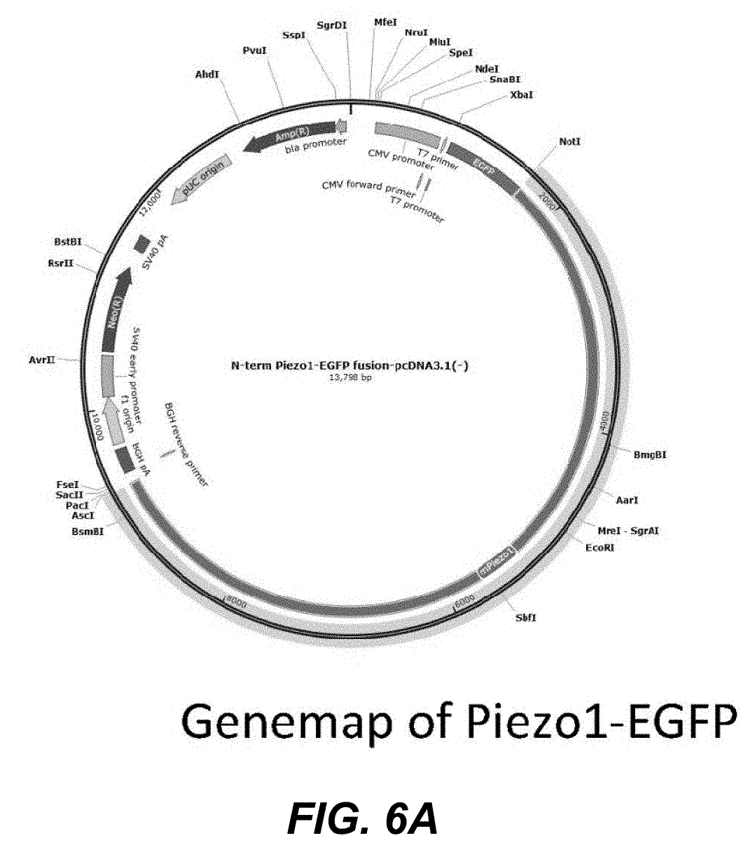

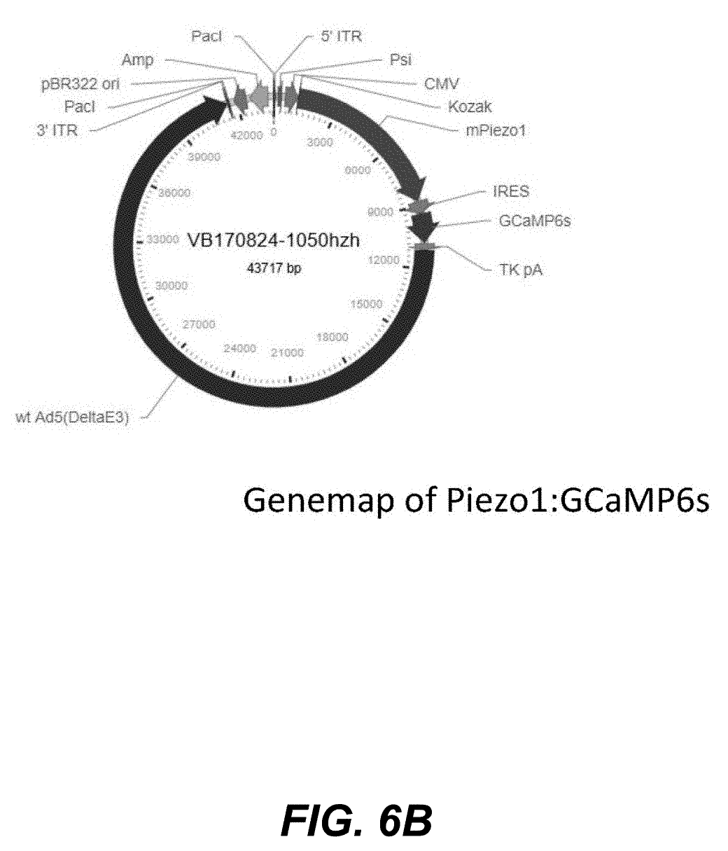

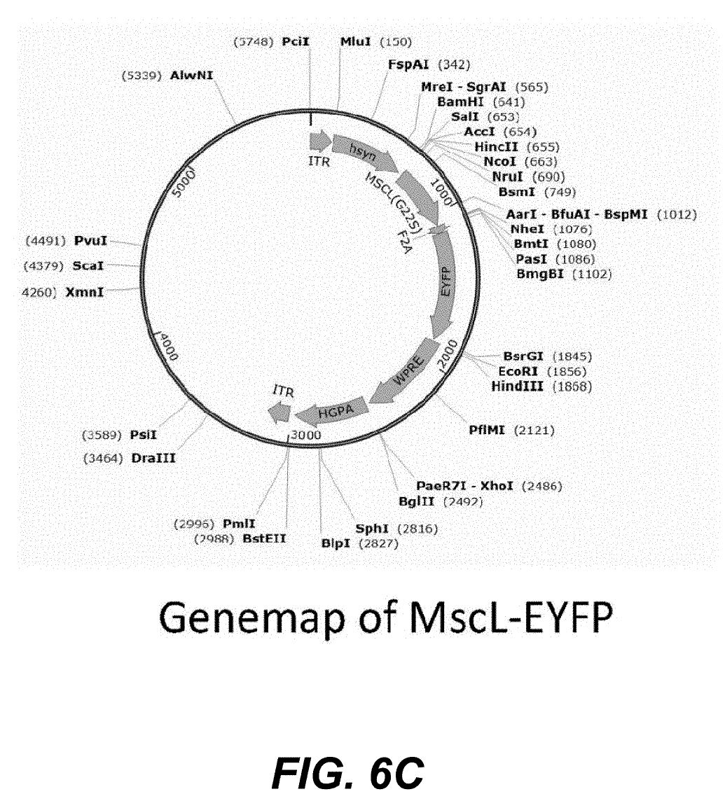

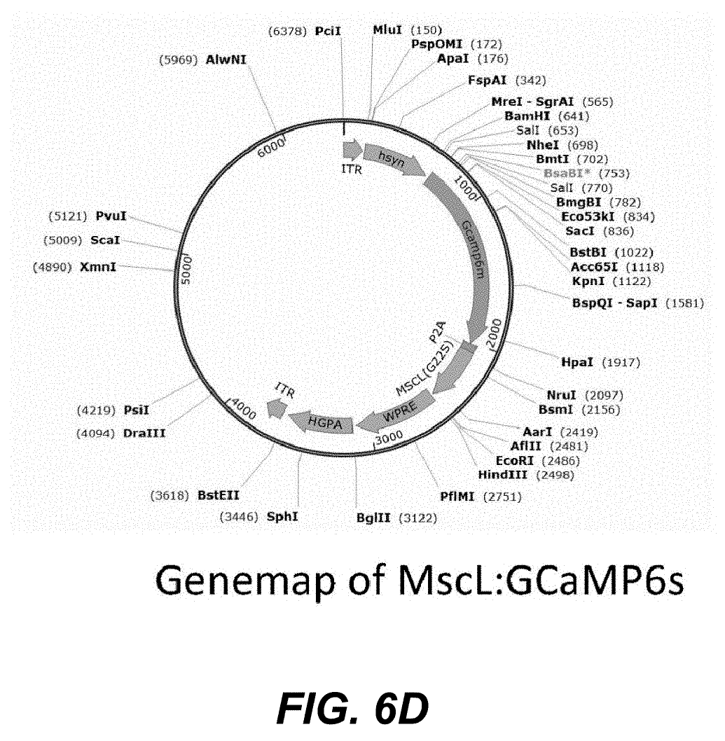

Also included for reference are gene maps for Piezo 1 EGFP (FIG. 6A) and Piezo1:GCaMP6s (FIG. 6B) and for MscL EYFP (FIG. 6C) and for MscL: GCaMP6s (FIG. 6D).

Variants may be substitutional, insertional or deletional variants prepared by site specific mutagenesis of nucleotides in the DNA encoding these proteins in predetermined locations. The variants may be created by using techniques such as PCR mutagenesis or other techniques well known in the art, to produce DNA encoding the variant, and thereafter expressing the DNA in recombinant cell culture.

While the site or region for introducing an amino acid sequence variation is predetermined, the mutation per se need not be predetermined. For example, in order to optimize the performance of a mutation at a given site, random mutagenesis may be conducted at the target codon or region and the expressed variants screened for optimal desired activity. Techniques for making substitution mutations at sites in DNA having a known sequence are well known, for example, PCR mutagenesis.

Substitutions, deletions, insertions or combinations may be used to arrive at a final derivative. Generally these changes are done on a few amino acids to minimize the alteration of the molecule.

The variants or derivatives typically exhibit the same qualitative activity as the piezo or MscL protein, although variants or derivatives also are selected to modify the characteristics as needed. Variants or derivatives can show enhanced ion selectivity, stability, speed, compatibility, and reduced toxicity. For example, the protein can be modified such that it can gain higher sensitivity, ion selectivity and be driven by different ultrasound frequency, so that one can do multiplexing control on different cells one demand.

It would be understood by a person of skill in the art that the proteins of the present disclosure can be coded for by various nucleic acids wherein each amino acid in the protein is represented by one or more sets of 3 nucleic acids (codons). Since many amino acids are represented by more than one codon, there is not a unique nucleic acid sequence that codes for a given protein.

Persons of skill in the art understand how to make a nucleic acid that can code for the proteins of the present disclosure by knowing the amino acid sequence of the protein. A nucleic acid sequence that codes for a polypeptide or protein is the “gene” of that polypeptide or protein. A gene can be RNA, DNA, or other nucleic acid than will code for the polypeptide or protein.

It is known by persons of skill in the art that the codon systems in different organisms can be slightly different, and that therefore where the expression of a given protein from a given organism is desired, the nucleic acid sequence can be modified for expression within that organism.

An aspect of the present disclosure provides a nucleic acid sequence that codes for an ultrasound activated cation channel protein that is optimized for expression with a mammalian cell. A preferred embodiment comprises a nucleic acid sequence optimized for expression in a human cell.

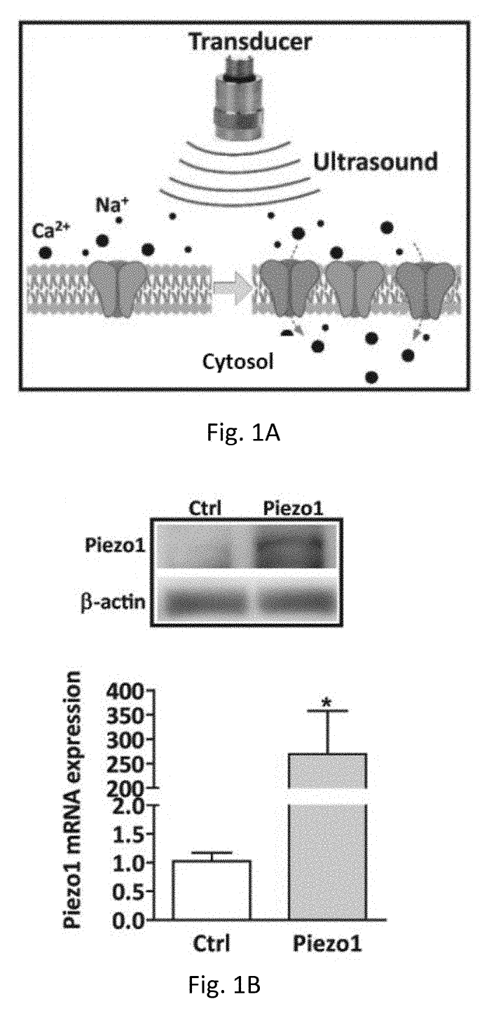

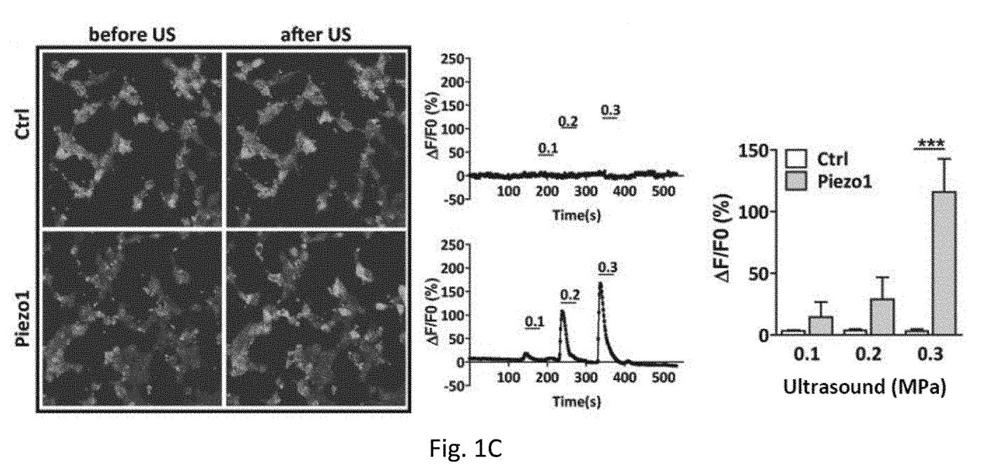

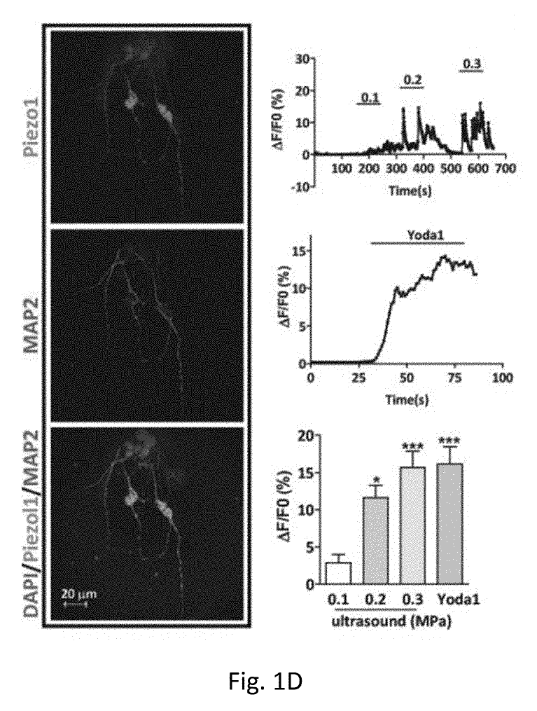

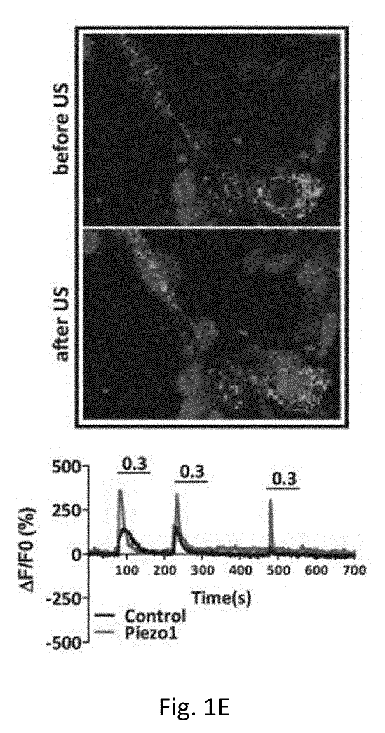

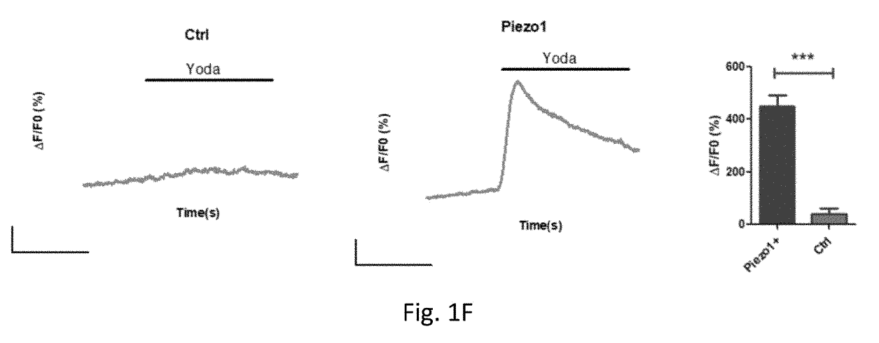

Piezo1 was overexpressed in 293T cells, known to have minimal expression of Piezo1 [5] by transfecting a plasmid encoding a Piezo1-EGFP fusion protein (as described in). The expression of Piezo1 in 293T cells was verified by Western blot and qPCR (FIG. 1B).

Hence, an aspect of the present disclosure is a fusion protein comprising an ultrasound-activated channel protein. It is well known in the art that fusion proteins can be made that will create a single protein with the combined activities of several proteins. In one embodiment, the fusion proteins can be used to target to specific cells or regions within cells, and provide for the ability of both independent stimulation of a mechanosensitive ion channel and the simultaneous monitoring of localization. The simultaneous stimulation and monitoring of localization can be carried out in many cell types including mammalian systems.

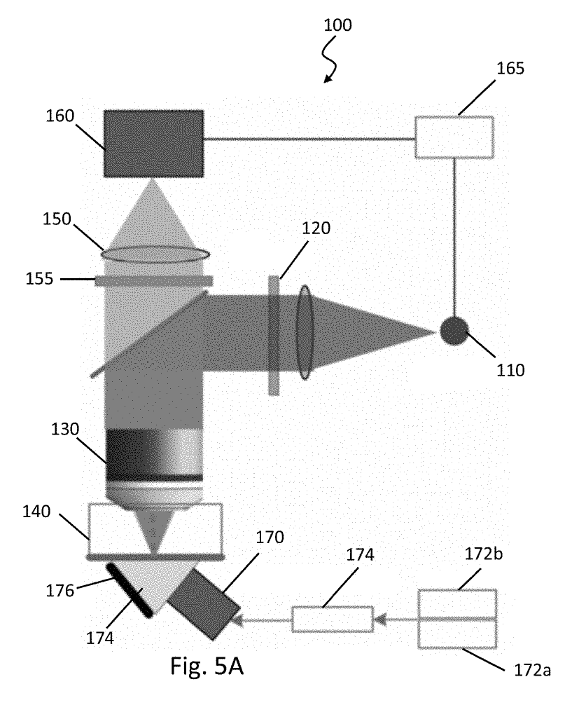

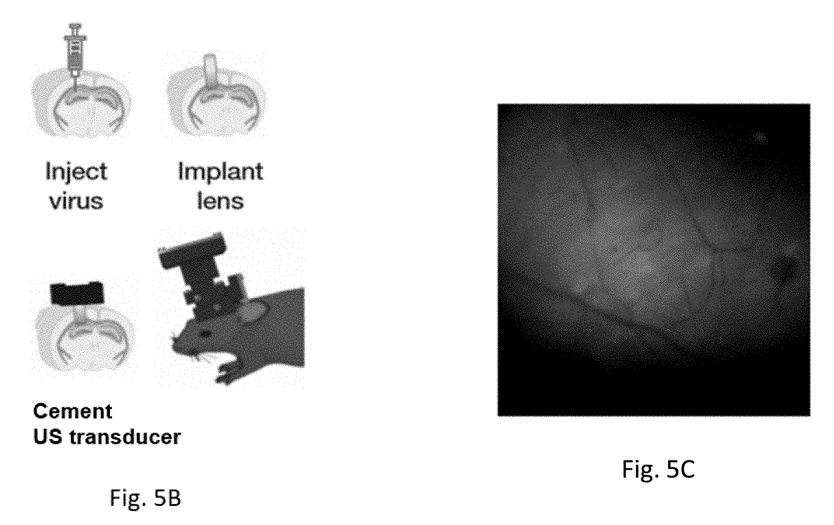

Using the in vitro setup (FIG. 5), we treated cells with 500 kHz ultrasound of 200 cycles, at 1 kHz pulse repetition frequency (PRF) with 200 tone bursts at 0.1, 0.2, and 0.3 MPa, respectively. A fluorescent calcium indicator was used to measure intracellular Ca2+. In response to Ultrasound Stimulation (US), dose-dependent Ca2+ influx was seen in cells treated with the Piezo1 plasmid, but not in cells transfected with the control plasmid.

Significant Ca2+ increase was observed in response to 0.3 MPa ultrasound (FIG. 1C), as well as to Yodal (FIG. 1F), a chemical agonist of Piezo1 (5). These results demonstrate that ultrasound stimulation can, therefore, specifically activate Piezo1.

Next, the effects of ultrasound on primary cortical neurons harvested from embryonic mouse brains to test the feasibility of stimulating live neurons with ultrasound. Primary neurons at day in vitro (DIV) 10 were seen to express Piezo1 endogenously and accumulated intracellular Ca2+ in response to ultrasound in a dose-dependent manner.

Ultrasound induces dose-dependent calcium influx in primary cortical neurons through Piezo1. Piezo1 expression in primary cortical neurons was confirmed by immunocytochemical staining; Green: Piezo1; Red: MAP2; DAPI: Blue. Ultrasound stimulation induced significant increase of intracellular calcium of the neuron, comparable to the effect of Yodal, a chemical agonist of Piezo1. * P<0.05, ***P<0.001, one-way ANOVA with post-hoc Tukey test.

Acoustic pressures of 0.2 and 0.3 MPa elicited significant responses, comparable to that produced by Yodal (FIG. 1F).

When transfected with the Piezo1-EGFP plasmid, primary neurons showed an even higher level of Ca2+ influx compared to untransfected cells (FIG. 1E), revealing that the Ultrasound-induced Ca2+ response is sensitive to the level of Piezo1 expression.

Hence overall, these results demonstrate that ultrasound stimulation can, therefore, specifically activate Piezo1; and that the level of ultrasound-induced Ca2+ response is sensitive to the level of Piezo1 expression.

Signalling

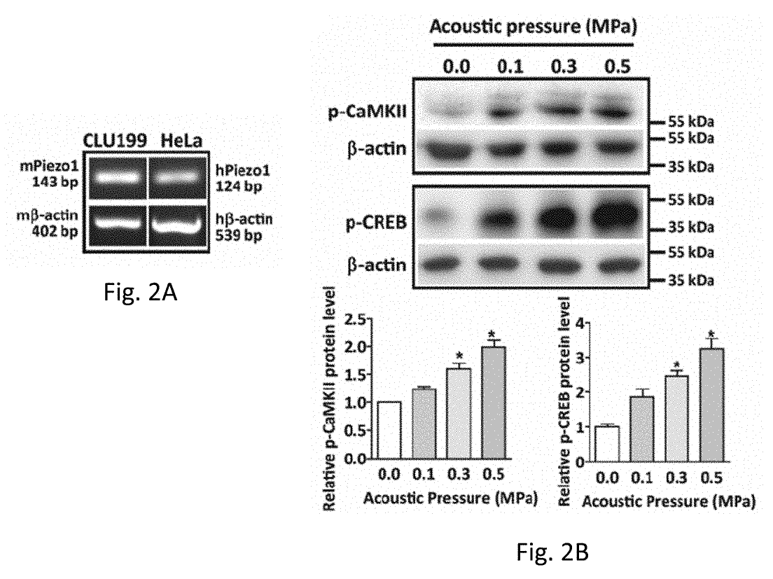

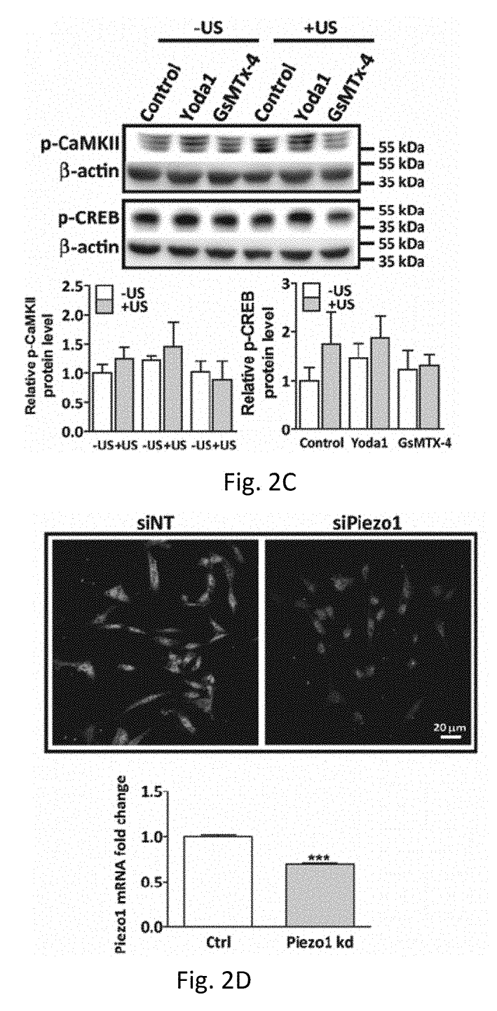

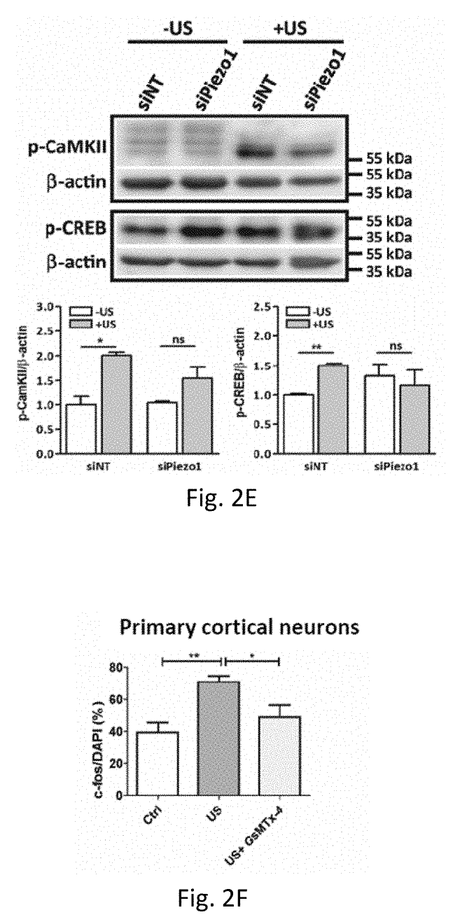

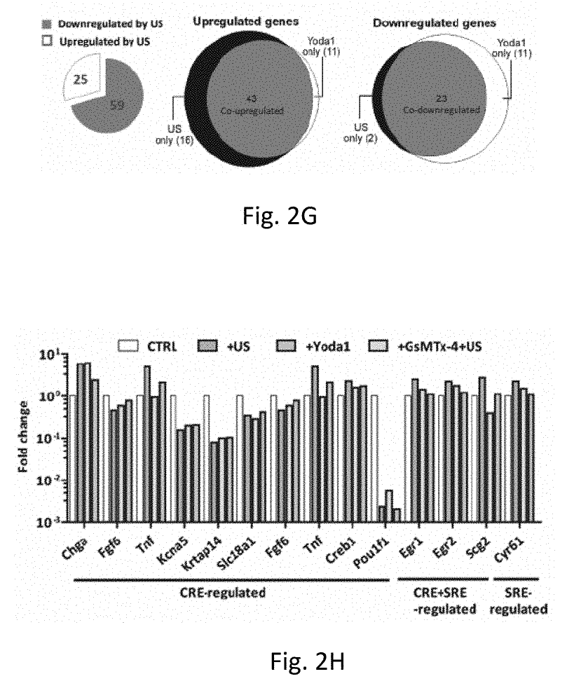

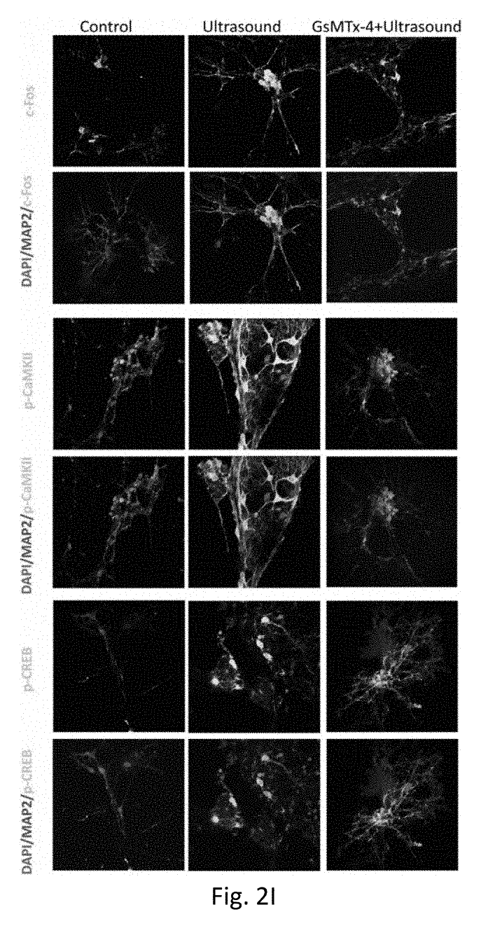

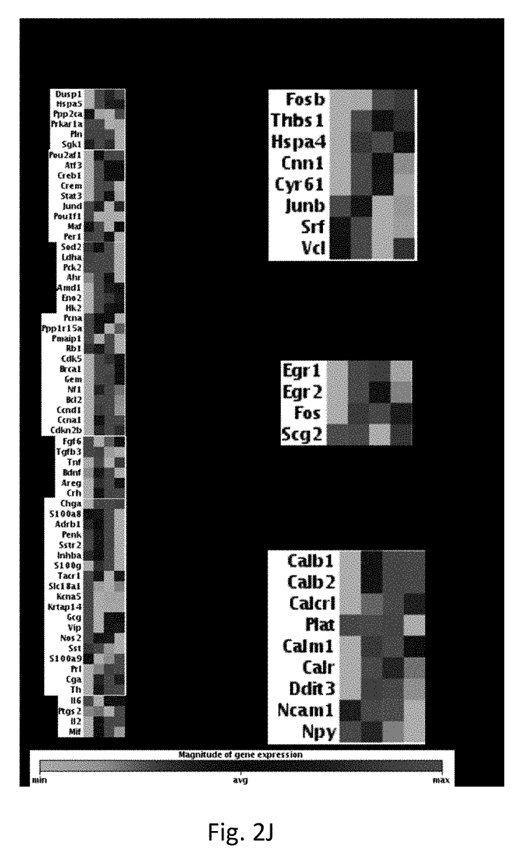

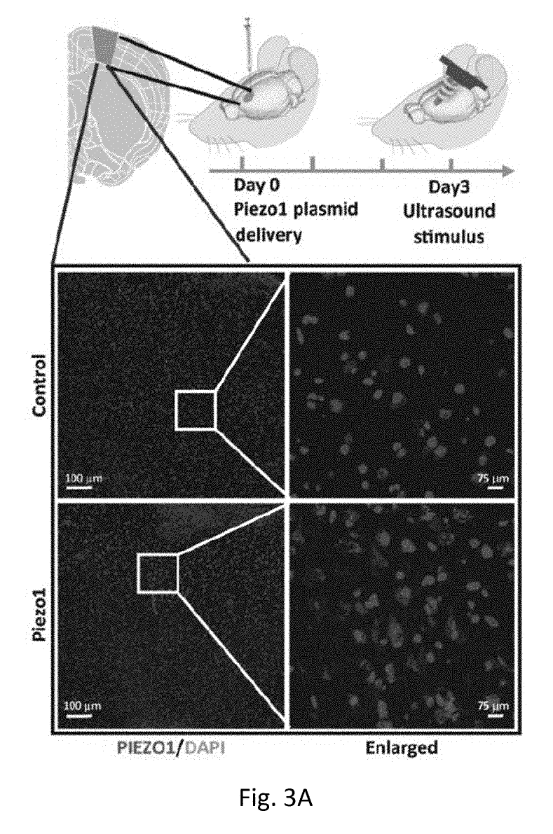

To understand the signalling implications of Piezo1-mediated ultrasound effects, the mouse hippocampal cell line mHippoE-18 (CLU199) was selected as an in vitro representation of normal neural cells. Expression of Piezo1 in CLU199 cells was confirmed through RT-PCR, with HeLa cells as a positive control (FIG. 2A).

To evaluate effects on cell signalling, the phosphorylated forms of the Calcium/calmodulin-dependent protein kinase type II (p-CaMKII) [6], and the transcription factor CREB (p-CREB) (28), both crucial players in Ca2+ signalling were analysed.

Ultrasound treatment increased the levels of both p-CREB and p-CaMKII in a dose-dependent manner, with 0.3 and 0.5 MPa inducing significant increases (FIG. 2B). 0.3 MPa ultrasound was then used to treat CLU199 cells with or without Yodal, or the Piezo1 blocker GsMTx-4 [7].

Ultrasound and Yodal alone increased the activation of both p-CaMKII and p-CREB, but with a pre-ultrasound GsMTx-4 incubation, both proteins were reduced to levels comparable to those in the untreated control (FIG. 2C).

siRNA was then employed to knock down Piezo1 in CLU199 cells (FIG. 2D). When treated with ultrasound post-transfection, levels of p-CaMKII and p-CREB in the non-targeting (siNT) condition increased, but with Piezo1 knockdown (siPiezol) levels of both proteins were reduced compared to siNT (FIG. 2E).

The activation of neurons by ultrasound in primary cortical neurons (FIG. 2J) was also tested by examining expression of the well-established neuronal activation marker c-Fos [8]. Ultrasound treatment significantly increased the level of c-Fos expression in the nuclei of primary neurons, and blocking Piezo1 with GsMTx-4 inhibited this effect (FIG. 2F), and affected the levels of p-CREB and p-CaMKII as well (FIG. 2G). Thus, the changes ultrasound induced through Ca2+-related signalling are Piezo1-specific and are also sensitive to the levels of Piezo1 expression.

Ultrasonic effects in CLU199 cells were explored using a qPCR array that probes 84 genes known to have Ca2+ responsive elements of various kinds.

The number of genes upregulated or downregulated by ultrasound treatment versus the untreated control were counted, and categorized according to their pathway of Ca2+-dependent regulation and their functions (FIG. 2G).

59 genes were found to be upregulated by ultrasound while 25 were downregulated (FIG. 2G).

Ultrasound and Yodal treatment co-upregulated 43 genes, while co-downregulating 23 (FIG. 2G), emphasizing the degree to which these effects of ultrasound are founded on the phenomenon of intracellular Ca2+ increase.

Initially, these results were filtered to identify genes whose expression levels were altered two times or more in either direction. These comprised 12 genes: the neurotransmitter-related Chga, Kcna5, Krtap14 and Slc18a, growth factors FGF6 and Tnf, transcription factors Creb1, Poulf1 (all CRE-regulated); Cyr61 (SRE-regulated); and the transcription factors Egr1 and Egr2 and the neurotransmitter-related Scg2 (all CRE- and SRE-regulated) (FIG. 2H).

The expression of c-Fos, p-CaMKII and p-CREB was investigated by immunocytochemical staining in primary cortical neurons untreated, treated by ultrasound or treated with GsMTx-4 (10 μM) followed by ultrasound. Each stimulus contained 200 cycles, at 1 kHz RPF with 200 NTB of 500 kHz ultrasound at 0.3 MPa and the results are shown in FIG. 2I.

A qRT-PCR array that explores genes that regulate calcium-related signalling (RT2 Pathway Finder) was used to evaluate how ultrasound treatment affects cellular signalling. Cells were also either pre-treated with GsMTx-4 (10 μM) before ultrasound or treated with Yodal (1 μM) by way of comparison. Results of the PCR were uploaded to Qiagen/SABiosciences online suite for data analysis, and clustergrams were generated using this software. Information about various subgroups of genes described here (FIG. 2J) were obtained from the product literature.

Ultrasound is hence seen to have important signalling effects via Ca2+ influx, which could affect properties of neurons such as plasticity, growth and neurotransmitter production and secretion.

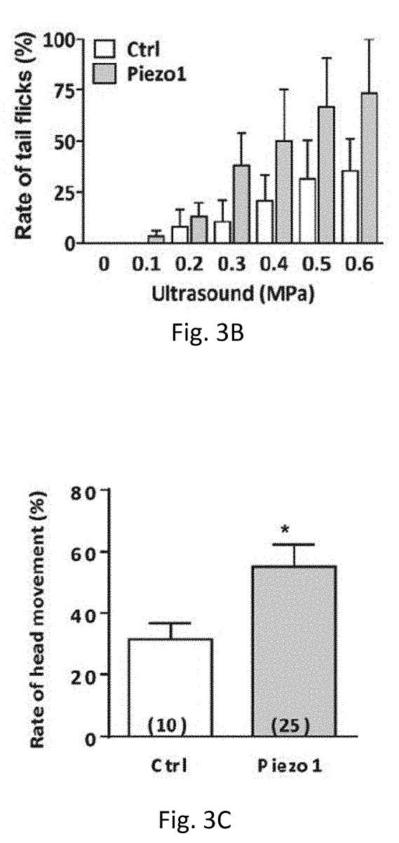

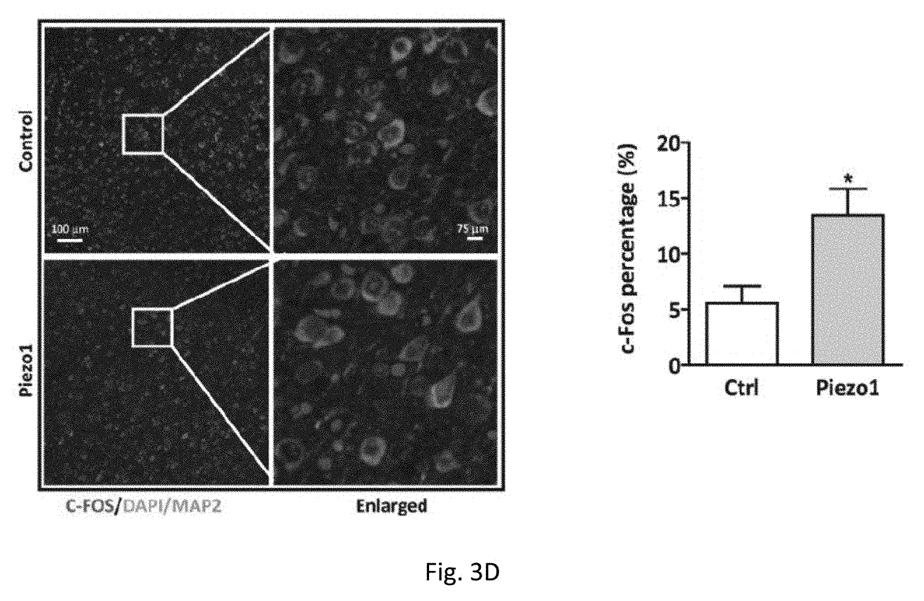

Piezo1 was shown to increase neuron sensitization to ultrasound (FIG. 3A, top) in vivo.

With well-characterized projections, like muscle in tail, paw and neck [9], the primary motor cortex (M1) is an established region in which to demonstrate the effects of a new neural control modality.

Piezo1 or control plasmids were injected into M1, and 3 days later this region was checked for Piezo1 expression (FIG. 3A(ii), showing higher Piezo1 expression in Piezo1-transfected mice. As shown in FIG. 3A(ii) Piezo1 was successfully expressed in M1 of mouse brains, 3 days after plasmid injection.

The anesthetized mice were exposed to ultrasound in a range of 0.1-0.6 MPa, and the motor responses were recorded and quantified with the number of tail flicks per ultrasound stimulus. Motor responses were found to be affected by the acoustic pressure of the ultrasound and were increased in the Piezo1-transfected mice compared to the control mice. At 0.3 MPa, tail flicks increased from 11% to 39% between the control and Piezo1-transfected mice, and this pattern increased to 36% vs 73% respectively at 0.6 MPa (FIG. 3B). At 0.3 MPa the tail flick rate by Piezo1-transfected mice was more than double that of the control mice, and we chose this condition to treat free-moving, plasmid-transfected mice.

The ultrasound-induced head movements per stimulus were also quantified when the mice were stationary (The Piezo1-transfected free-moving mice exhibited a significantly higher head movement rate than the control mice (51% vs 31%) upon ultrasound (FIG. 3C). Additionally, ultrasound stimulation in the M1 region urged Piezo1 transfected mice to run around with no such effects on control mice. Neural excitation was examined using c-Fos expression as an indicator. Neural activity triggered by ultrasound in M1 was significantly higher in Piezo1-transfected mice, with 13.4% c-Fos-positive cells, compared to only 5.6% in control mice (FIG. 3D). In this way, upregulating Piezo1 in mouse brains enabled increased neuronal sensitivity to the excitatory effects of ultrasound stimulation in vivo.

To demonstrate the functional applicability of these results to other mechanosensitive ion channels we have conducted similar experiments using Mscl-G22s EYFP in neuronal tissue, and noted similar behaviours as discussed above in relation to FIG. 3A to FIG. 3D.

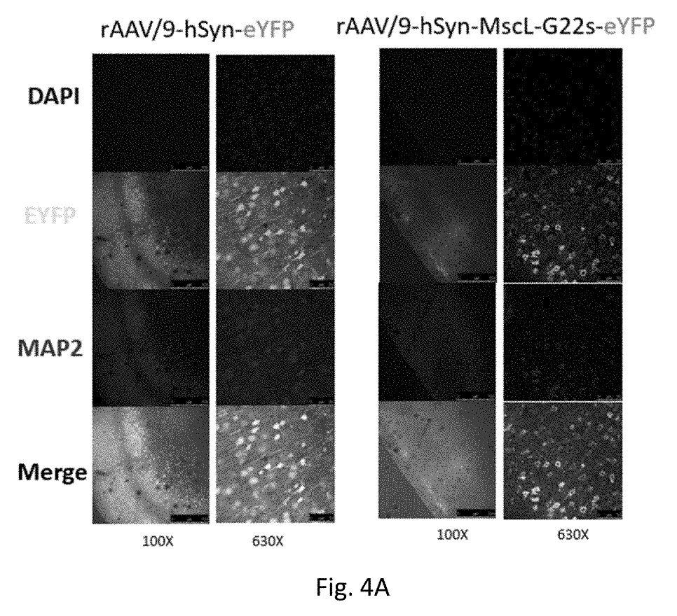

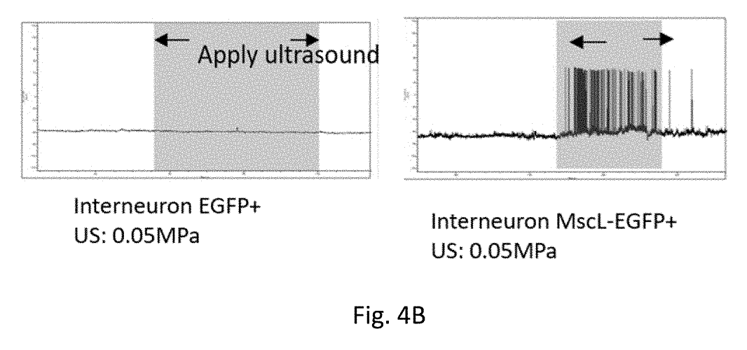

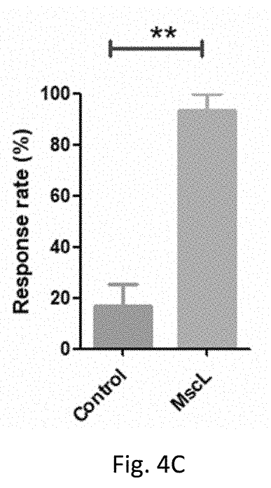

FIG. 4A depicts two groups of pictures of brain neural tissue, on the left is the control group under 100× and 630× magnification, showing the results after transfection with the raaV and Enhanced Yellow Fluorescent Protein to demonstrate the success of the transfection.

On the right hand side, neural tissue which has been infected with rAAV including MscL channel protein depicted. The visible EYFP on the cell membrane clearly demonstrate that MscL can be successfully expressed in neural brain tissue using this strategy.

FIG. 4B depicts patch clamping results of control neurons when receiving ultrasound stimulation of 0.05 MPA; and compares this to the behaviour in vivo of MscL expressing neurons with similar stimulation. It is apparent that even under conditions of relatively low pressure ultrasound used in the study that ultrasound stimulation can activate the MscL expressing neurons.

Similar to the results depicted in FIG. 3C in relation to Piezo1, FIG. 4C depicts the results of movement of head/tails in free moving mice exposed to ultrasound in a range of 0.3 MPa which have been transfected with MscL-G22s.

The motor responses were recorded and quantified with the number of tail flicks/head twitches per ultrasound stimulus. Motor responses were found to be affected by the acoustic pressure of the ultrasound and were increased in the Piezo1-transfected mice compared to the control mice.

At 0.3 MPa the tail flick rate by Mscl-G22s-transfected mice was more than five times that of the control mice as expected.

Taken together, the present disclosure demonstrates the feasibility of using Piezo1 and MscL-G22s as an effective mediator of neural activity and signalling when treated with ultrasound. It is expected and early studies confirm that similar behaviour and circuit level specificity could also be achieved with other mechanosensitive ion channels such as ENac, CFTR, TRPV-1, allowing pass differentiations and different sensitivity.

FIG. 5A depicts an exemplary calcium imaging system 100 consisting of a modified upright epifluorescence microscope.

The excitation light was generated by a dual-color LED 110, filtered by 488 nm bandpass filters 120 and delivered via the objective lens 130 to the sample in the sample holder 140 for illuminating the calcium sensor.

The fluorescence signals from the cells were collected by a water immersion objective (UMPlanFLN, Olympus) 130, filtered by a filter wheel with green (525 nm) or red (633 nm) channels 155 LPF; focussed by a tube lens 150 and captured by a sCMOS camera 160 (ORCA-Flash4.0 LT Plus C114400-42U30, Hamamatsu) operated by a controller 165. To minimize phototoxic effects, the LEDs were triggered at 1 Hz and synchronized with sCMOS time-lapse imaging.

The ultrasound stimulation system consisting of a commercial transducer 170 (I7-0012-P-SU, Olympus), two function generators 172a,172b, and a power amplifier 174 (Electronics and Innovation, A075) to produce 200 tone burst pulses at a center frequency of 500 kHz and a repetition frequency of 1 kHz with a duty cycle of 40%. The output intensity was limited to 0.1-0.6 MPa. These parameters are similar to which has been reported to effectively evoke behaviour responses (3). To deliver ultrasound, a triangle waveguide 174 was attached to the ultrasound transducer 170 and placed under the culture dish at a 45-degree angle to the horizontal axis. The other site of the waveguide was mounted with an acoustic absorber 176 to minimize acoustic reverberation. During calcium imaging, the cells were placed in a buffer solution with 130 mM NaCl, 2 mM MgCl2, 4.5 mM KCl, 10 mM Glucose, 20 mM HEPES, and 2 mM CaCl2, pH 7.4.

Advantageously, the benefit of providing stimulation using ultrasound stimulus in conjunction with optical imaging to observe the response means that the system avoids deficiencies in prior art arrangements of optogenetics using light stimulation in conjunction with optical imaging to observe the effects of such stimulation.

Referring to FIG. 5B, there is shown an exemplary schematic representation of how calcium imaging can be conducted in vivo simultaneously with the application of ultrasound to understand the impact of opening particular mechanosensitive ion channels in specific portions of the brain.

The system comprised an ultrasound stimulation system and mini-epifluorescence imaging system. The excitation light from LED was delivered into a specific brain region by a GRIN lens while the excited fluorescence from GCaMP6s were collected and filtered into a sCMOS camera. The combination of the mini-fluorescence imaging system with ultrasound stimulation system was achieved by replacing the normal baseplate by an ultrasound transducer with a hole in the centre for insertion of GRIN lens. The transducer baseplate can be plan, or focused as align with the optical focusing or has some sort of down-stream or up-stream relation to calcium imaging.

FIG. 5C is an exemplary image captured by this arrangement showing that fluorescence imaging of GCaMp6s overexpressing neurons in vivo in the presence of ultrasound baseplate. It demonstrates clear fluorescence signal and the ultrasound baseplate itself will not influence the imaging quality.

In an aspect of the disclosure there is provided an ultrasound-activated channel protein that is non-toxic in the cells in which it is expressed. Preferably, the ultrasound-activated channel proteins of the present disclosure do not change electrical properties, or otherwise adversely affect cellular survival. Preferably, the ultrasound activated channel proteins of the present disclosure do not alter membrane resistance of the cells in the absence of ultrasound. Further, the channels do not lead to apoptosis in the cells, nor lead to the generation of aberrant nuclei.

Preferably, in the absence of ultrasound, the presence of the piezol or MscL protein does not alter cell health or ongoing electrical activity. Preferably, the presence of piezol or MscL protein creates no significant long-term alterations in the properties of neurons expressing the protein.

By manipulating expression of mechano-sensitive ion channels such as Piezo1 and MscL-G22 in the desired neurons, we demonstrate a new approach for selective, non-invasive brain stimulation.

The various consequent effects of this strategy, regulating genes with functions from transcription control to neurotransmitter production, indicate ultrasound is a modality with wide-ranging effects that could be applied therapeutically to a variety of different conditions. This approach could potentially be utilized for probing brain functions and treating disorders, which could be identified through more in-depth research about the treatment's effect on behaviour.

The methods of controlling cell properties of the present disclosure will result in the ability to probe function in intact neural circuits, allowing for the understanding of the role of particular neurons in animal models including of learning, memory and motor coordination. This will enable the discovery of drugs capable of modulating whole-circuit function, essential for the addressing of complex neurological and psychiatric diseases. For the first time, genetically-targeted neurons within animals will be addressable by ultrasound allowing examination behavioral or circuit-dynamics function and allow for observed normal and dysfunctional behaviours.

In some embodiments it would be appreciated that the specific spatial and temporal control of neurons that is provided by the present disclosure could potentially provide the ability to target neural circuits non-invasively for understanding brain functions and dysfunctions, as well as providing a treatment strategy, which can hopefully translate to clinical practice.

Control over synaptic events through regulation of mechanosensitive channels expressed endogenously or introduced therein enables the potential for control over cell plasticity, learning and memory. Potentially similar such mechanisms can be utilised to explore and/or treat neurological disorders such as Parkinsons disease, Alzheimers disease, diabetes, neuro-immuno modulation.

In one aspect of the research detailed herein about specific spatial and temporal control over the activity of mechano-sensitive ion channels in relation to signalling, specific control over the levels of the transcription factor CREB (p-CREB) were demonstrated.

cAMP responsive element binding protein (CREB) is a known nuclear protein that modulates the transcription of genes with cAMP responsive elements in their promoters. Increases in the concentration of either calcium or cAMP can trigger the phosphorylation and activation of CREB. Genetic and pharmacological studies in mice and rats demonstrate that CREB is required for a variety of complex forms of memory, including spatial and social learning, thus indicating that CREB may be a universal modulator of processes required for memory formation. (1) Hence the ability to control Ca²⁺ levels and CREB through the use of ultrasound as demonstrated by the results of the present disclosure provide potential to control the underlying biochemical bases for memory; important in treatment of disease and ordinary function.

Furthermore, control of CREB has been specifically implicated in the activation of specific ensembles of neurons in a specific region of the mice brain to increase the recall of fear memory (see e.g. 13);

CREB has also been implicated as a treatment target of a number of age related cognitive defects, (11) which could be usefully further studied with the method of the present disclosure.

It is envisaged that indirect control of other types of ion channels in the cell could also be achieved by modulating the activity of such genes as TRPV1,TRPV4, Enac, CFTR, Piezo2.

### EXAMPLES

1. Cell Culture

293T cells were purchased from ATCC. The embryonic mouse hippocampal cell line mHippoE-18 (referred to in the text as “CLU199”) was purchased from Cedarlane Laboratories. 293T and CLU199 cells were maintained in Dulbecco's Modified Eagle Medium (DMEM) (high glucose and no sodium pyruvate), supplemented with 10% fetal bovine serum, 100 units/mL penicillin and 100 μg/mL streptomycin (all from Gibco), inside a humidified incubator 37° C. with 5% CO2. For experiments requiring transfected cells, cells were seeded in 35 mm dishes or collagen-I-coated (Corning) glass coverslips (5 μg/cm2), at 1.5×106 cells per dish, allowed to grow overnight, and treated with ultrasound the next day.

2. Primary Cortical Neuron Harvest

Primary cortical neurons from embryonic mice brains were harvested as described previously with slight modifications. Briefly, pregnant mice were sacrificed at E16.5-E17, and brains from the embryonic mice were collected. They were then dissected under a microscope to separate the cortex from the other brain mass. Cortical cells were dispersed and seeded into culture dishes or collagen-coated glass coverslips (4 μg/cm2). At DIV 10, the cells were used for further experiments.

3. Plasmid Transfection and siRNA Reverse-Transfection

pcDNA3.1 plasmids, both the control plasmid and the one containing Piezo1-EGFP, were provided by Dr. Ardem Patapoutian (Scripps Institute). Transfections on 293T cells were performed using 1 μL of Lipofectamine™ LTX Reagent with PLUS™ Reagent (Invitrogen) with 500 ng of plasmid DNA in Opti-MEM (Gibco) per 35 mm dish or 6-well plate. The media was changed to DMEM+FBS after 24 hours. Transfected cells were used for imaging and ultrasound stimulation 48 hours after transfection. Primary cortical neurons were transfected on DIV 7 following the same protocol as for 293T cells. Transfected primary neurons were used for imaging and ultrasound stimulation 72 hours after transfection.

SMARTpool ON-TARGETplus Piezo1 siRNA (L-061455-00-0005) and ON-TARGETplus Non-Targeting Pool (D-001810-10) siRNA were obtained from Dharmacon. Transfection complexes were prepared by incubating siRNA with Lipofectamine RNAiMAX (Invitrogen) in Opti-MEM for 5 minutes at room temperature. 300 μl of complexes added per 35 mm dish or collagen-coated coverslips (5 μg/cm2). 1.5×106 cells were added per dish in fully-supplemented DMEM such that the final siRNA concentration was 125 nM. Cells were incubated for 48 hours post-transfection, and then used for further experiments.

4. Calcium Imaging

Cells were loaded with the fluorescent calcium indicator Cal590 (AAT Bioquest), according to the manufacturer's instructions. A customized calcium imaging and ultrasound stimulation system (FIG. 4) was utilized for the study.

5. Ultrasound Treatment of Cells

CLU199 cells or primary cortical neurons at DIV 10 were treated with ultrasound at acoustic pressures of 0.1, 0.3 or 0.5 MPa for 20 minutes with 20 s interval inside a humified incubator, 37° C., 5% CO2. The ultrasonic stimulation system and treatment parameters are the same as those described in section 4. Yodal (Tocris Bioscience) was used as a Piezo1 agonist with a concentration of 1 μM, while GsMTx-4 (Abcam) was used as a Piezo1 blocker at a concentration of 10 μM. Yodal was added to culture media, followed by a 20-minute incubation or immediate ultrasound treatment. GsMTx-4 was added to culture media, allowed for 30 minutes incubation, and followed by a further 20-minute incubation or ultrasound treatment.

6. Western Blotting

Cells were lysed with RIPA buffer (EMD Millipore), supplemented with 1× Halt™ Protease and Phosphatase Inhibitor Cocktail (Thermo Fisher Scientific) on ice, centrifuged to clear the lysate, and protein concentrations measured using the Bio-Rad protein assay. 30 or 50 μg total protein per well was loaded on 10% SDS-PAGE gels, electrophoresed, and transferred to methanol-activated PVDF membranes. Membranes were blocked with 3% BSA in Tris-buffered-saline+0.05% Tween-20 (TBST). Membranes were incubated in primary antibody diluted in 5% BSA in TBST overnight, washed with TBST and incubated in secondary antibody solutions, diluted in blocking buffer, for 1 hour at room temperature. Membranes were then washed, signals developed with SuperSignal™ West Pico (cat. no. 34078) or Pierce ECL (cat. no. 32106) chemiluminescent substrates according to the manufacturer's instructions, and imaged using the Bio-Rad ChemiDoc MP system.

Primary antibodies used were phospho-CREB (cat. no. 9198), CREB (cat. no. 9197), phospho-CaMKII (cat. no. 12716) and CaMKII-pan (cat. no. 4436) all from Cell Signaling Technology and diluted at 1:500 and β-actin (A1978, Sigma-Aldrich) at 1: 2,000 was the loading control. Secondary antibodies used were goat-anti-rabbit IgG (H+L) HRP (cat. no. 31460) and goat-anti-mouse IgG (H+L) HRP (cat. no. 31430) from Thermo Fisher Scientific, diluted at 1: 5,000 in blocking buffer.

Protein levels were quantified through image densitometry using ImageJ. Protein levels were expressed as a fold change compared to the untreated control, an average ±SEM of at least three independent experiments, except in FIG. 2E. Statistical significance was calculated using a two-tailed unpaired student's t-test and P values below 0.05 were considered significant.

7. RNA Extraction and Reverse-Transcription

Cells were lysed with RNAiso Plus (Takara) and RNA was extracted from these lysates using RNA Direct-zol columns (Zymo Research) according to the manufacturer's instructions, including a DNAse incubation step to eliminate genomic DNA from the final product. RNA was quantified using a NanoVue spectrophotometer (GE Healthcare Life Sciences), and 1 μg of total RNA was reverse-transcribed using the Transcriptor First Strand cDNA Synthesis Kit (Roche).

8. Semi-Quantitative PCR and Real-Time qPCR

1 μl cDNA from CLU199 cells was mixed with 2×PCR Premix Ex Taq (Takara) (final concentration 1×), forward and reverse primers (mouse Piezo1 and β-actin, final concentration 200 nM) and H₂O to a final reaction volume of 20 μl. PCR was performed on a Bio-Rad DNA Engine thermal cyler, for 25 cycles, Ta 56° C. PCR product was loaded on a 2% agarose gel, electrophoresed, visualized in an AlphaImager HP (ProteinSimple) UV Transilluminator. The resulting bands were quantified using image densitometry in ImageJ.

For real-time qPCR, 1 μl cDNA from plasmid-transfected 293T or siRNA-transfected CLU199 cells was mixed with appropriate forward and reverse primers (final concentration 250 nM), 2×SYBR Green Premix Ex Taq (Takara) and H₂O to a final volume of 10 μl. PCR was performed on Applied Biosystem 7500 Fast Real-Time PCR system (Thermo Fisher Scientific). Results are expressed as a fold change compared to the appropriate control, mean±SD of 3 independent experiments. Primer sequences were as follow:

Mouse β-actin:
F-
(SEQ ID NO: 3)
AGG GTG TGA TGG GAA TG,
R-
(SEQ ID NO: 4)
TGG CGT GAG GGA GAG CAT AG, 402 bp;
human β-actin:
F-
(SEQ ID NO: 5)
GTG GGG CGC CCC AGG CAC CA,
R-
(SEQ ID NO: 6)
CTC CTT AAT GTC ACG CAC GAT TTC, 539 bp;
mouse Piezo1:
F-
(SEQ ID NO: 7)
GCA GTG GCA GTG AGG AGA TT,
R-
(SEQ ID NO: 8)
GAT ATG CAG GCG CCT ATC CA, 143 bp;
human Piezo1:
F-
(SEQ ID NO: 9)
ATCGCCATCATCTGGTTCCC,
R-
(SEQ ID NO: 10)
TGGTGAACAGCGGCTCATAG, 124 bp;
mouse GAPDH:
(SEQ ID NO: 11)
F-AAC GAC CCC TTC ATT GAC,
R-
(SEQ ID NO: 12)
TCC ACG ACA TAC TCA GCA C, 190 bp.

9. qPCR Array

CLU199 cells seeded in 35 mm dishes were treated with ultrasound, Yodal or GsMTx-4 as appropriate. Total RNA was collected as described in section 7, and the concentration measured on NanoDrop™ One (Thermo Fisher Scientific). Samples with 260/280>1.8 and 260/230>2.0 were run on a 1% agarose gel containing bleach to check for RNA integrity (2). Samples with good integrity were reverse-transcribed using the RT2 First Strand Kit (Qiagen), using 1 μg total RNA, according to the manufacturer's instructions. RT product was diluted as described in the manufacturer's protocol, and mixed with RT2 SYBR® Green qPCR Mastermix (Qiagen). This solution was then loaded into appropriate wells of the RT² Profiler™ PCR Array Mouse cAMP/Calcium Signaling PathwayFinder (cat. no. PAMM-066Z), centrifuged and cycled according to the manufacturer's instructions on the QuantStudio™ 7 Flex Real-Time PCR System (Life Technologies). Ct values were calculated using the Quantstudio™ software, exported into a spreadsheet and uploaded to SABiosciences' online array data analysis software (accessible at: http://pcrdataanalysis.sabiosciences.com/per/arrayanalysis.php). Results were normalized to three housekeeping genes (Gapdh, Gusb and Hsp90ab1). Resulting clustergrams and a spreadsheet containing the analyzed results were obtained from this software. The number of genes that were upregulated (fold change >1.00) or downregulated (fold change <0.50) compared to the untreated control were calculated, and the list of genes whose expression was altered by ultrasound treatment by more than 2 times was collected. The results were categorized according to the nature of the gene's regulation and the function of the encoded protein, according to information provided in the PCR array's product literature and in a paper, that also used this array.

10. Immunocytochemical Fluorescent Staining

CLU199 cells and primary neurons at DIV 10, including treated with (+) or without (−) ultrasound, were fixed using 4% paraformaldehyde+PBS and permeabilized using 0.1% Triton X-100+PBS. Cells were blocked using 3% normal goat serum+3% BSA in PBST, and incubated overnight in primary antibodies diluted in 5% BSA+0.05% sodium azide. Secondary antibody incubation was performed the next day, diluted in 3% BSA in PBST for one hour at room temperature. Cells were washed, coverslips dried, and mounted on glass slides using small drops of Prolong Diamond Antifade Mountant with DAPI (Life Technologies), and allowed to cure in the dark at room temperature overnight. All wash steps were performed using PBS. Coverslip edges were then sealed using transparent nail enamel, and imaged using a confocal laser scanning microscope (TCS SP8, Leica). The number of c-Fos+ cells were determined by counting the number of DAPI and c-Fos signals co-located with DAPI from 10 FOVs photographed per condition. Results were expressed as a percentage of c-Fos versus DAPI signals, and were subjected to a one-way ANOVA followed by a post-hoc Tukey test. P values below 0.05 were considered significant.

Primary antibodies, used at a dilution of 1:200, were phospho-CREB, phospho-CaMKII and c-Fos (cat. no. 2250) all from Cell Signaling Technology, Piezo1 (15939-1-AP, Proteintech Group), and MAP2 (MAB3417, EMD-Millipore). Secondary antibodies, used at a dilution of 1:1,000, were goat anti-rabbit IgG (H+L), Alexa Fluor 488 (A-11008) and goat anti-mouse IgG (H+L), Alexa Fluor 555 (A-21422) from Invitrogen.

11. Animal Care

Male, 8-week old, C57BL/6J mice, were used for ultrasound stimulation with 6 in an anesthetized group and 6 in a free-moving group. Mice were housed under standard housing condition with food and water available ad libitum. Animal use and care were performed following the guideline of the Department of Health—Animals (Control of Experiments) of the Hong Kong S.A.R. government.

12. Plasmid Injection and Ultrasound Transducer Mounting

Mice were anaesthetized by intraperitoneal injection of Ketamine and Xylazine (100 mg/kg and 10 mg/kg respectively) followed by removing the skin above M1 area. Using the stereotaxic apparatus, a hole was drilled to allow pipette injection (AP=1.34, ML=1.50, DV=0.75). 1 μg plasmid mixed with in vivo-jetPEI® (Polyplus Transfection) was injected at a rate of 0.5 μl/min, and followed by a 10-minute pause. The pipette was then retracted slowly, including a 5-minute pause at the halfway point. To stimulate anesthetized mice, a transducer (I7-0012-P-SU, Olympus) was placed above the mouse head and directed to M1 region coupled by ultrasound gel. To stimulate free-moving mice, a holder for a customized wearable ultrasound transducer was mounted to the mouse brain. Afyoda

ter 3 days recovery, an ultrasound transducer at a center frequency of 775 kHz was clicked to the pre-mounted holder and locked tightly with preloaded ultrasound gel for stimulation.

13. Ultrasound Stimulation and Behavior Recording

Anesthetized mice, were given 10-15 min to reach to an appropriate anesthesia plane. The mice were treated with 500 kHz ultrasound of 200 tone burst pulses and a repetition frequency of 1 kHz with 40% duty cycle and 0.1-0.6 MPa acoustic pressure. The number of tail flicks versus the number of ultrasound stimuli were recorded. The results were analyzed with an unpaired two-tailed t-test, and P-values below 0.05 were considered significant.

For the free-moving experiment, mice were given 4 minutes to calm down and their motor responses following ultrasound stimulus at 0.3 MPa were recorded with a camera. Ultrasound stimuli were performed in batches of 10 each, each referred to as 1 “trial” in the text, and mice were allowed one minute of rest between trials. The number of head swings following every ultrasound stimulus was counted per trial. The number of head movements versus the number of ultrasound stimuli per trial were then analyzed with a two-tailed unpaired t-test, and P values below 0.05 were considered significant.

14. Immunohistochemical Fluorescent Staining

Mice were sacrificed 90 minutes after ultrasound treatment and perfused with PBS, followed by 4% paraformaldehyde (PFA) (cat. no. P1110, Solarbio) in PBS. After dissection, brains were post-fixed overnight in 4% PFA and then dehydrated in 15%, 20% and 30% sucrose diluted in PBS for the following 3 days. Starting from the injection plane, 60 continuous coronal brain slices at a thickness of 30 μm were collected. Slices were blocked using 1% normal goat serum+5% BSA+PBS, and incubated overnight in primary antibody solution diluted in 5% BSA+0.05% sodium azide. Slices were then washed, and incubated with secondary antibodies diluted in PBS for 1 hour at room temperature. Slices were then washed, coverslips dried, and mounted on glass slides using small drops of Prolong Diamond Antifade Mountant with DAPI, and allowed to cure in the dark at room temperature overnight. Coverslip edges were sealed using transparent nail enamel, and imaged using a confocal laser scanning microscope (TCS SP8, Leica). 3 ROIs were chosen from a slice and the number of cells showing blue (DAPI) and red (c-Fos) signals were counted using ImageJ, and the number of c-Fos signals were expressed as a percentage of the number of DAPI signals. The number of c-Fos positive cell vs DAPI positive cell were analysis by unpaired two-tailed t-test and P values lower than 0.05 were considered significant.

Primary antibodies used were c-Fos (2250, CST, dilution 1:50), Piezo1 (15939-1-AP, Proteintech Group, dilution 1:50), and MAP2 (PA1-16751, Invitrogen, dilution 1:100). Secondary antibodies, used at a dilution of 1:500, were goat anti-rabbit IgG (H+L), Alexa Fluor 488 (A-11008, Invitrogen) and goat anti-mouse IgY (H+L), Alexa Fluor 633 (A-21103, Invitrogen).

The above embodiments are described by way of example only. Many variations are possible without departing from the scope of the disclosure as defined in the appended claims.

Although a variety of examples and other information was used to explain aspects within the scope of the appended claims, no limitation of the claims should be implied based on particular features or arrangements in such examples, as one of ordinary skill would be able to use these examples to derive a wide variety of implementations. Further and although some subject matter may have been described in language specific to examples of structural features and/or method steps, it is to be understood that the subject matter defined in the appended claims is not necessarily limited to these described features or acts. For example, such functionality can be distributed differently or performed in components other than those identified herein. Rather, the described features and steps are disclosed as examples of components of systems and methods within the scope of the appended claims.

### REFERENCE

1. W. Legon, T. F. Sato, A. Opitz, J. Mueller, A. Barbour, A. Williams, W. J. Tyler, Transcranial focused ultrasound modulates the activity of primary somatosensory cortex in humans, Nat. Neurosci. 17, 322-329 (2014).
2. B. Martinac. Mechanosensitive ion channels: an evolutionary and scientific tour de force in mechanobiology, Channels (Austin, Ill.,) 6, 211-213 (2012).
3. B. Coste, M. Schmidt, T. Earley, S. Ranade, M. Petrus, A. Dubin, A. Patapoutian, Piezo1 and Piezo2 Are Essential Components of Distinct Mechanically Activated Cation Channels, Science 330, 55-60 (2010).
4. C. Grienberger, A. Konnerth, Imaging Calcium in Neurons, Neuron 73,862-885 (2012).
5. R. Syeda, J. Xu, A. E. Dubin, B. Coste, J. Mathur, T. Huynh, J. Matzen, J. Lao, D. C. Tully, I. H. Engels, H. Michael Petrassi, A. M. Schumacher, M. Montal, M. Bandell, A. Patapoutian, Chemical activation of the mechanotransduction channel Piezo1, Elife 4 (2015), doi:10.7554/eLife.07369.
6. T. Yamauchi, Neuronal Ca2+/calmodulin-dependent protein kinase II—discovery, progress in a quarter of a century, and perspective: implication for learning and memory, Biol. Pharm. Bull. 28, 1342-1354 (2005).
7. R. Gnanasambandam, C. Ghatak, A. Yasmann, K. Nishizawa, F. Sachs, A. S. Ladokhin, S. I. Sukharev, T. M. Suchyna, GsMTx4: Mechanism of Inhibiting Mechanosensitive Ion Channels, Biophys. J. 112,31-45 (2017).
8. M. Sheng, M. E. Greenberg, The regulation and function of c-fos and other immediate early genes in the nervous system, Neuron 4,477-485 (1990).
9. P. P. Ye, J. R. Brown, K. B. Pauly, Frequency dependence of ultrasound neurostimulation in the mouse brain, Ultrasound Med. Biol. 42, 1512-1530 (2016).
10. Silva, Alcino & H. Kogan, Jeffrey & W. Frankland, Paul & Kida, Satoshi. (1998). Creb and memory. Annual review of neuroscience. 21. 127-48. 10.1146/annurev.neuro.21.1.127.
11. Yu, X. W., Oh, M. M., & Disterhoft, J. F. (2017). CREB, cellular excitability, and cognition: Implications for aging. Behavioural Brain Research, 322, 206-211. DOI: 10.1016/j.bbr.2016.07.042
12. Yii X W, Curlik D M, Oh M M, Disterhoft J F. CREB overexpression in dorsal CA1 ameliorates long-term memory deficits in aged rats. ELife. 2017 Jan. 4; 6. pii: e19358. doi: 10.7554/eLife.19358. PubMed PMID: 28051768; PMCID: PMC5214885.
13. Han, J., Josselyn, S. A, Kim, J., Kwon, J., E Kim, H. (2014). Memory recall and modifications by activating neurons with elevated CREB. Nature Neuroscience, 17, 65-72.

1. A method for reversibly stimulating at least one or more neuronal cells, said method comprising activating mechanosensitive ion channels of at least one or more neuronal cells expressing said channels by exposing said cells to an ultrasound stimulus.

2. The method for reversibly stimulating at least one or more neuronal cells according to claim 1 wherein the mechanosensitive ion channels are selected from the group comprising Piezo 1, MscL-G22s, CFTR.

3. The method for reversibly stimulating at least one or more neuronal cells according to claim 1 wherein mechanosensitive ion channels expressed in the neuronal cells are introduced to or increased in said cells by introducing a recombinant nucleic acid encoding the mechanosensitive ion channels into said cells or precursors thereof.

4. The method for reversibly stimulating neuronal cells according to claim 3 wherein the mechanosensitive ion channels are introduced to or increased in the neuronal cells by transfection with a plasmid or infection with a virus containing a promoter sequence coupled with a mechanosensitive ion channel gene.

5. The method for reversibly stimulating neuronal cells according to claim 1 wherein the ultrasound stimulus is in the range of 0.1 MPa to 0.6 MPa, and more preferably in the range of 0.2 MPa to 0.5 MPa, and more preferably 0.5 MPa.

6. The method for reversibly stimulating neuronal cells according to claim 5 wherein the ultrasound stimulus applied is at about 500 kHz ultrasound of at least 200 cycles, at about 1 kHz pulse repetition frequency (PRF) with 200 tone bursts at about 0.1, 0.2, and 0.3 MPa.

7. The method for reversibly stimulating neuronal cells according to claim 1 wherein the method further comprises monitoring the intracellular cation activity.

8. The method for reversibly stimulating neuronal cells according to claim 7 wherein the method further comprises the step of monitoring the intracellular Ca2+ activity.

9. The method for reversibly stimulating neuronal cells according to claim 8 wherein the step of determining intracellular Ca2+ activity is performed by any one or more of fluorescent staining, levels of Calcium/calmodulin-dependent protein kinase type II (p-CaMKII) and the transcription factor CREB (p-CREB).

10. The method for reversibly stimulating neuronal cells according to claim 8 wherein optical monitoring of intracellular Ca2+ activity in vivo in an animal is conducted at least during the application of the ultrasound stimulus to the animal.

11. The method for reversibly stimulating neuronal cells according to claim 10 wherein optical monitoring is performed by an image capturing device engageable with the animal.

12. A method for studying specific spatial and temporal activity of neuron cells in vivo in a mammal comprising:
(a) transfecting neuronal cells with plasmids having one or more sequences encoding a mechanosensitive ion channel into neuronal cells to produce infected neuronal cells expressing increased numbers of said channel; and
(b) exposing said cells to an ultrasound stimulus whilst monitoring the intracellular Ca2+ activity therein.

13. A method of increasing neuron sensitivity to ultrasound by overexpression therein of at least one or more proteins selected from the group comprising piezol, MscL-G22s, CFTR, wherein the overexpression is by introducing genetic material encoding the at least one or more proteins in the neuronal cells by transfection with a plasmid or a virus wherein said plasmid or virus has a sequence encoding the at least one or more proteins.

14. A system for studying specific spatial and temporal activity of neuron cells in vivo in a mammal, the system comprising:
a population comprising a plurality of neuronal cells genetically modified so as to express therein an increased number of mechanosensitive ion channels;
an ultrasound source for activating mechanosensitive ion channels engageable with the mammal and proximal to said cells;
an optical imaging source arranged adjacent the transfected neuronal cells for providing images of the neuronal cells at least during activation of the mechanosensitive ion channels by the ultrasound source.

15. The system for studying specific spatial and temporal activity of neuron cells in vivo in a mammal as claimed in claim 14 wherein the ultrasound source activates the mechanosensitive ion channels in the population.


SEQUENCE LISTING (12 sequences)

SEQ ID NO: 1 - DNA, 7644 bp, Mus musculus
atggagccgcacgtgctgggcgccgggctctactggctgttgctgccctgcacgctcctggcggcctccctgttacgcttcaatgctctctcgctggtctatttgctgtttctactgctgctgccctggcttccaggcccctcaagacacagcataccaggtcacacaggtcgcctgctccgtgcactactctgcctcagcctcctcttcctggtggcccaccttgcctttcagatatgcctacacaccgtgcctcacctggaccagtttctgggacaaaacggtagcctttgggtgaaggtgtctcaacacataggggttacgaggctggacctgaaggacatctttaacaccaccaggctggtagcacctgacctgggagtgctgctggcgtcctccctttgccttggcctctgtggacgcctcacgaggaaagccgggcagagtcggcgcacccaggagctgcaggatgatgacgatgacgatgatgacgacgatgaagacatagatgctgccccagccgtggggctgaagggagcccctgccctggcaaccaaacgcaggctgtggctggcctcccgcttccgggtcacggcccactggctgctgatgacctctggacggacgctggtcattgtgttgctggccctggcaggcatagcccacccttcggccttctccagcatctacctggtggtgttcctggccatctgcacctggtggtcctgtcactttcctctcagccccctgggctttaacaccctctgtgtcatggtgagctgttttggtgccggccatctcatttgcctatactgctatcagacaccatttatccaggacatgttaccccctgggaacatctgggccaggctatttggtctcaagaacttcgtagacctccctaactactccagccccaacgccctggtactcaacactaagcacgcctggcccatctatgtgagtcccggaatcctgctgctgctatattacacagccacctctctcctgaagctccacaagagctgtccctcagagctgaggaaggagacacccagggaggatgaggagcatgagctggaactggaccacctggagccagagccccaggctagggacgccacccagggtgagatgcccatgaccacggaacctgaccttgacaactgcaccgtgcatgtactaaccagccagagccctgtccgccagcgtccagttcgccccaggctggctgagctgaaagagatgtcaccgctacatggcctgggccacctcatcatggaccagagctatgtgtgtgccctgattgccatgatggtgtggagcatcatgtaccacagttggctgaccttcgtcctgctgctctgggcctgcctcatctggacggtgcgtagccgtcaccagctggctatgctctgctcgccctgcatcctgctgtatgggctgacgctctgctgcctgaggtatgtgtgggccatggaacttcctgagctgcccaccaccctgggccctgtcagcctgcaccagttgggactggaacacacacgctacccttgcctggacctcggtgccatgctcctctatctgctcacattctggctccttctgcgtcagttcgtgaaggagaagctgctgaagaagcaaaaggtgcctgcggcgctgctggaggtcacggtggccgacactgagcccacacagacccagacgctgctgcggagcctgggggagctggtcaccggcatctacgtcaaatactggatctatgtgtgcgccggcatgttcattgtggtcagcttcgccggccgcctggtggtctacaaaatcgtctacatgttcctcttcctgctgtgcctcaccttgttccaggtctactacaccctgtggaggaagctgctacgtgtcttctggtggctcgtggtggcctatacaatgctcgtgctcatcgctgtgtacaccttccagttccaggacttccccacctattggcgcaacctcacgggcttcacagacgagcagttgggcgacctgggcctggagcagttcagtgtgtcggagctcttttccagtatcctcatccctggcttcttcctgctggcctgcatcctgcagctgcactacttccacagaccgttcatgcagctcactgacctggagcacgtgccgccaccaggcacccgccaccctcgatgggctcacaggcaggatgcagtgagcgaggcccctctgcttgagcatcaggaggaagaggaagtcttcagggaagatgggcagagcatggatgggccccaccaggccacacaggtccctgagggtacggccagcaagtggggcctggtggctgaccggctgctggacctagcggccagcttctcggctgtcctcacccgaatccaggtgttcgttcggcgcttgctagaacttcacgtcttcaagctggtggccctctacactgtctgggtggccctgaaagaagtgtctgtgatgaacctgctgctggtggtgctatgggccttcgccttgtcctatccgcgcttccggcccatggcttcctgcctgtccaccgtgtggacctgtatcatcattgtatgcaagatgctctatcagctcaagattgtcaacccgcatgagtactccagcaactgcactgagcccttccccaacaataccaacttgcagcctttggagatcaaccagtctttgctgtaccgtggccctgttgaccctgccaactggtttggggtgcggaagggttaccccaacttgggctatatccagaaccacctgcagatccttctgttgctggtgtttgaggccgtggtgtaccggcgccaagagcactaccgccggcagcaccagcaggcccctctgcccgcccaggctgtgtgcgcagatggcacccgccagaggttggaccaggacctacttagctgcctcaagtatttcatcaacttcttcttctacaaattcgggctggagatctgcttcttgatggccgtgaatgtgattgggcagcgtatgaacttcatggtgatcttacacggttgctggttggtggccatccttacacgccggcgccgtgaggccatcgcccgcctctggcctaactactgtctgttcctcacgctgttcctgctgtaccagtacctgctgtgtttgggcatgccccctgcactgtgcattgactatccatggcgctggagcaaggccatccccatgaattccgccctcatcaagtggctgtacctacctgacttcttcagagcccccaactccaccaaccttatcagtgacttcctcctgctgctttgcgcctcccagcagtggcaggtcttctcagcggagcgaacggaggagtggcaacgcatggcgggcatcaacactgaccacctggagcccctgcgtggggagcccaaccctatacccaacttcatccactgcaggtcctatctggatatgctgaaggtggccgtcttccgctacctgttctggctggtgctcgttgtggtgtttgttgcgggggccacccgcataagcatcttcgggctggggtacctgctagcctgcttctacctgctgctgtttggcactaccctgctgcagaaggacacgcgagcccagctcgtgctgtgggactgcctcatcctctataatgtcactgtcatcatctctaagaatatgctgtcgctcctgtcctgtgtcttcgtggagcaaatgcagagcaacttctgctgggtcatccagctcttcagcctcgtgtgcacagtcaaaggctactatgatcccaaagagatgatgaccagggaccgggactgcctgctgcctgtggaggaggccgggatcatctgggacagtatctgcttcttcttcctgctcttgcaacggcgcatctttctcagccactacttcctgcatgtcagcgctgacctgaaagccacagccctgcaggcatccaggggctttgccctctacaatgcagccaacctgaagagcatcaacttccatcgccagattgaggagaagtccctggcccagctgaaaagacagatgaagcgcatccgtgccaaacaggagaagtacaggcagagccaggcaagtcgtggccaactccagtccaaagaccctcaggatcccagccaggagccagggcctgacagcccagggggctcctccccgccacggagacagtggtggcgcccctggctggaccacgccacagtcatccactctggcgactacttcctgtttgagtcagatagcgaggaggaagaggaggccctacctgaggaccccaggcctgcagctcagagtgccttccagatggcataccaggcatgggtaaccaatgcccagacagtgctgaggcagcgtcgggagcgggcacggcaggagcgggcagagcagctggcttctggaggtgacttgaacccagatgtggaaccagtagatgtcccagaagatgagatggcaggccgtagccacatgatgcagcgtgtgctaagcaccatgcagttcctgtgggtgctgggccaggccacggtagacgggctgacgcgctggctgcgtgcattcacgaagcaccaccgcaccatgagcgatgtgctgtgcgcagagcgctacctgctcacccaggagcttcttcgggttggagaggtacgccgaggtgtgctggaccagctttatgtgggtgaagatgaggccacattgtcaggtcccgtggagacccgggatggacccagcacagcctcaagtgggctgggagccgaagagcctttgagtagcatgacagacgacaccagcagccccctgagcacaggctataacacccgcagtggcagtgaggagattgtcaccgacgctggggacctccaggctgggacctccctgcacggctcccaagagcttttagccaatgctcgtacccggatgcgcacggccagcgagctgctactggataggcgcctgcatatccctgagctggaggaggccgagcggtttgaggcacagcagggccggactctgcggctgctcagggctgggtaccagtgcgtggcggcacactcggagctgctctgttacttcatcatcatccttaaccacatggtgacagcctcggctgcctccctggtgctgcccgtgcttgtgttcctgtgggccatgctgaccatcccgaggcctagcaagcgcttttggatgacagctatcgtcttcactgaggtcatggtggtcaccaaatacctgttccagttcggcttcttcccctggaacagctacgttgtgctgcggcgctatgagaacaagccctacttccctccgcgaatcctgggccttgagaaaacggacagctacatcaagtatgacctggtgcagctcatggccctcttcttccaccgctcgcagctactgtgttatggcctctgggaccatgaggaggatcgctatcccaaggaccattgcaggagtagtgtgaaggaccgggaggccaaggaagagccagaagctaagctggaatcgcagtctgagacaggcactgggcatcccaaggagccagtgttggccggtactcccagggaccacatccaagggaaaggaagtattagatccaaggatgttatccaagatcccccagaggaccttaagccccggcacacgaggcacatcagcatacgcttcaggaggcgcaaggagactccaggacccaaaggaacagcagtcatggagactgagcacgaggagggagaaggaaaagaaactacagagagaaagaggccgcgtcacactcaagaaaaatcgaagtttcgggagagaatgaaggcagctgggcgccggctgcagagcttctgtgtgtcactggcccagagcttctaccaacccttgcagcgtttcttccatgacattctgcacacaaagtaccgggcggccaccgacgtctacgccctcatgttcctggccgatattgtcgacatcatcatcatcatctttggtttttgggcttttgggaagcactctgcagccacagacattgcatcctcgctgtcagatgaccaggtgccacaggccttcctgttcatgctgctggtccagtttggcaccatggtcatcgaccgtgccctctacctgcgcaagactgtcctgggaaagctggcctttcaggtggtcctggtggtggcgattcacatctggatgttctttatcttaccggctgtcactgagaggatgttcagccagaatgcggtggcacagctgtggtacttcgtcaagtgcatttactttgccctgtccgcctaccagatccgctgtggctaccccacccgtatcttgggcaacttcctcaccaagaaatacaaccatctaaacctcttccttttccaggggttccgtctagtgccgttcctggtggagctgcgggccgtcatggactgggtgtggaccgacaccacgctgtccctgtccaactggatgtgtgtggaagacatctatgccaacatctttatcatcaagtgcagccgagagacagagaagaaatacccccagcccaaggggcagaagaagaagaaaattgtcaagtatggtatgggaggcctcattatcctcttcctcatcgccatcatctggttccctctgctcttcatgtcactgatccgctctgtggtcggggtcgtcaaccagcccattgatgtcaccgtcaccctcaagctaggcggctatgagccactgttcaccatgagcgcccagcagccatccattgtgccattcacaccccaggcctacgaggagctgtcccagcagtttgacccctatccactagccatgcagttcattagccagtacagtcctgaggacatcgtcactgcacagatcgagggcagctcgggggcgctgtggcgcatcagcccacccagccgagcccagatgaagcaggagctgtacaacggcacagccgacattacactgcgctttacctggaatttccaaagggacctggccaagggtggcactgtggagtatactaatgagaagcacaccttggagctggcccccaacagtacggcacgaaggcagctggcccaactgctcgagggcagacctgaccagtcagtggtcattccccatctcttccccaagtacattcgtgctcccaatgggcctgaagccaaccctgtgaagcagctgcagccagatgaggaagaggactaccttggtgtgcgcatccagctgcggagggagcaagtgggcacaggggcctctggggagcaagcgggcaccaaggcctccgacttcctcgagtggtgggtcatcgagctgcaggactgcaaggctgactgcaacctgctgcccatggtcatcttcagtgacaaggtcagcccacctagcctgggcttcctggccggctacgggattgtggggctgtacgtctccatcgtgctggtggttggcaagtttgtgcggggcttcttcagcgagatctctcactccatcatgttcgaggaactgccgtgtgtggaccgcatcctcaagctgtgccaggacatcttcttggtgcgcgagacccgggagctggagctggaggaggagctatacgccaagctcatcttcctgtaccgatctccagagaccatgattaagtggacacgtgagagggagtag

SEQ ID NO: 2 - DNA, 405 bp, Escherichia coli
agcattattaaagaatttcgcgaatttgcgatgcgcgggaacgtggtggatttggcggtgagtgtcattatcggtgcggcattcgggaagattgtctcttcactggttgccgatatcatcatgcctcctctgggcttattaattggcgggatcgattttaaacagtttgctgtcacgctacgcgatgcgcagggggatatccctgctgttgtgatgcattacggtgtcttcattcaaaacgtctttgattttctgattgtggcctttgccatctttatggcgattaagctaatcaacaaactgaatcggaaaaaagaagaaccagcagccgcacctgcaccaactaaagaagaagtattactgacagaaattcgtgatttgctgaaagagcagaataaccgctct

SEQ ID NO: 3 - DNA, 17 bp, Artificial Sequence
  Primer sequence
agggtgtgatgggaatg

SEQ ID NO: 4 - DNA, 20 bp, Artificial Sequence
  Primer sequence
tggcgtgagggagagcatag

SEQ ID NO: 5 - DNA, 20 bp, Artificial Sequence
  Primer sequence
gtggggcgccccaggcacca

SEQ ID NO: 6 - DNA, 24 bp, Artificial Sequence
  Primer sequence
ctccttaatgtcacgcacgatttc

SEQ ID NO: 7 - DNA, 20 bp, Artificial Sequence
  Primer sequence
gcagtggcagtgaggagatt

SEQ ID NO: 8 - DNA, 20 bp, Artificial Sequence
  Primer sequence
gatatgcaggcgcctatcca

SEQ ID NO: 9 - DNA, 20 bp, Artificial Sequence
  Primer sequence
atcgccatcatctggttccc

SEQ ID NO: 10 - DNA, 20 bp, Artificial Sequence
  Primer sequence
tggtgaacagcggctcatag

SEQ ID NO: 11 - DNA, 18 bp, Artificial Sequence
  Primer sequence
aacgaccccttcattgac

SEQ ID NO: 12 - DNA, 19 bp, Artificial Sequence
  Primer sequence
tccacgacatactcagcac